SEQUENCE LISTING (80 sequences)

SEQ ID NO: 1 - DNA, 2049 bp, Arabidopsis thaliana
  CDS (77)..(1810)
gcaaaacataagcgaggcagagtcttcagaaaacttacctgctctaaacaacgcctccgtgtccaagctcacttcaatggcgttacagctactcctagctgtattctcttgtgttcttcttcttcctcaacctgcatttgggattacaaggcattatacgctggaaatcaaaatgcagaacgtaacacgtctttgccacacaaagagccttgtttctgtaaacgggcagtttccaggtcctaagcttattgctagagaaggtgaccaggttctgatcaaagtcgttaatcaagtgccaaacaacatctctctccactggcatgggatccggcaattacgaagtggttgggctgatggtccagcctatataacccaatgtcctattcagacaggacaaagctatgtttacaactataccattgttggtcaacgaggcactctgtggtaccatgctcacatttcatggctaagatcaacagtctatggtccacttatcatccttcccaaacgcggagttccttacccgtttgctaaacctcacaaagaagttcccatgatctttggggagtggttcaacgcagacactgaggcaatcatccgccaagcaacccaaacaggaggtggtcccaatgtctctgatgcttacacgataaacgggcttcctggtccattatacaactgctccgcaaaagatacattcagactgagagtgaagccaggaaaaacataccttctcaggctaatcaatgctgcacttaatgacgagctctttttcagcatcgcaaatcacacggttacggttgttgaagctgatgcgatctatgttaagccatttgagactgaaaccatcttaattgctcctggtcagaccacaaacgtcctgctgaagactaaatctagttatccgagtgcctccttcttcatgactgctagaccatacgtcacaggtcaaggaacttttgataactctacagttgctggaatcttagaatatgaaccacctaaacagaccaaaggtgctcactcaaggacctctatcaaaaatcttcaactcttcaaaccgatactccctgctctaaacgatacaaattttgctaccaagttcagtaataagctacgcagcctgaacagcaaaaactttccagcaaacgtgcctctgaatgttgatcggaagttcttcttcacagtaggactgggaacaaacccgtgcaatcataagaataaccagacatgccaaggtcctactaacaccacaatgtttgctgcttcaatcagtaacatttcattcacaatgccaacaaaagctctccttcaatctcactattctgggcaatctcatggagtgtattccccaaaattcccatggagtcccattgtcccttttaactacacaggcactccacctaacaatactatggttagtaacgggacaaacttgatggttctaccttataacaccagtgtggagttggtgatgcaagacactagcattcttggcgcagaaagccatcctcttcatcttcatgggttcaacttctttgttgttggccaagggtttgggaatttcgacccgaacaaggatcctagaaacttcaaccttgttgacccaatagagaggaacacagtcggtgtgccatctggtggatgggctgctattcgattccttgcagataacccaggagtgtggttcatgcactgtcacttggaagtgcataccagttggggtctgaggatggcttggcttgttcttgatggagataaacctgatcagaaacttcttcctcctcctgcagacttgcccaaatgctgagaattctttccccatgattcaagtcctttttctttttgtttcttgtctcttctttcttacaagcttcgcacatttttttcgtttttttcctcggccatttatcggtttgacatgaggtcaattcctgttcaataatttgacatgaggtcaatttctttacaattatttactgttgcacagctctgttttctgcaagtgtagatctatgtaaacccaaaactaatatatatacacaatct

SEQ ID NO: 2 - protein, 577 aa, Arabidopsis thaliana
MALQLLLAVFSCVLLLPQPAFGITRHYTLEIKMQNVTRLCHTKSLVSVNGQFPGPKLIAREGDQVLIKVVNQVPNNISLHWHGIRQLRSGWADGPAYITQCPIQTGQSYVYNYTIVGQRGTLWYHAHISWLRSTVYGPLIILPKRGVPYPFAKPHKEVPMIFGEWFNADTEAIIRQATQTGGGPNVSDAYTINGLPGPLYNCSAKDTFRLRVKPGKTYLLRLINAALNDELFFSIANHTVTVVEADAIYVKPFETETILIAPGQTTNVLLKTKSSYPSASFFMTARPYVTGQGTFDNSTVAGILEYEPPKQTKGAHSRTSIKNLQLFKPILPALNDTNFATKFSNKLRSLNSKNFPANVPLNVDRKFFFTVGLGTNPCNHKNNQTCQGPTNTTMFAASISNISFTMPTKALLQSHYSGQSHGVYSPKFPWSPIVPFNYTGTPPNNTMVSNGTNLMVLPYNTSVELVMQDTSILGAESHPLHLHGFNFFVVGQGFGNFDPNKDPRNFNLVDPIERNTVGVPSGGWAAIRFLADNPGVWFMHCHLEVHTSWGLRMAWLVLDGDKPDQKLLPPPADLPKC

SEQ ID NO: 3 - DNA, 2021 bp, Arabidopsis thaliana
  CDS (81)..(1757)
cgaaaacagagagaaaaagactaaaaacacagtttaagaagaaggagagatagagagagaagagaaagatagagagggagatggggtctcatatggtttggtttctatttcttgtatccttcttctctgtgttcccagctccatctgagagcatggttcgccactacaagtttaacgttgtaatgaagaacgtgactagattatgctcaagcaagccaaccgtgaccgtcaacggtagatatccaggtcccacaatctacgcacgagaagatgacacgttgctcatcaaagtcgttaatcacgtcaagtacaacgtctccatccactggcacggtgtgagacaagtgagaacgggatgggctgatgggcctgcttacataactcagtgcccgatccagcctggtcaagtctacacatacaactacactttgaccggccaacgcggaacgctctggtggcacgctcatatcctctggctccgagccactgtttacggtgcattggtcatccttcccaaacgcggtgttccctatcctttccccaaacccgacaatgagaaagtcatcgttctaggtgaatggtggaaatcggatactgaaaatattattaatgaggcgcttaagtctggattagcccctaatgtctctgactctcacatgatcaacggacacccaggcccagttagaaactgtccatctcaaggttacaaactgtcagtagagaatggcaaaacctatctgctacgactagtcaacgctgcacttaatgaagaactctttttcaaagtcgccggccatattttcacggtggtagaagtagacgcagtctatgttaaaccgttcaagaccgacaccgtccttatagcccccggtcaaaccaccaacgtcctcctaaccgcctcaaaatccgccgggaaataccttgtaaccgcttctcctttcatggacgccccaatcgcggtggacaacgtaaccgccaccgcaactgttcattactcgggaacactctcctcctccccaacaatcctcacccttcctcccccgcaaaacgctacttccatagccaacaacttcacaaactctcttcgtagtctcaactccaagaagtaccctgctcttgtcccgaccaccatcgaccaccacctcttcttcaccgtcggccttgggctaaacgcatgccctacttgcaaggccggaaacggaagccgtgtcgtggctagcatcaacaatgtaaccttcattatgcctaaaaccgctttgctcccggctcattacttcaacacaagtggagttttcacgacagactttcccaagaatccaccacacgttttcaactacagcggaggatcagtcacgaacatggccacagaaaccggcacaaggctctacaagctaccgtataacgccactgttcagcttgtccttcaagataccggcgtcatagcgccagagaaccatccagtacatcttcacggttttaacttttttgaagtcggtcgtggattaggtaacttcaactccacgaaagacccaaaaaacttcaatttggtagatccggttgagaggaacacaatcggagttccatccggtggatgggtcgtcatcagattcagagcagataatcccggggtttggttcatgcattgtcacttggaggtacacacgacgtggggattaaagatggctttcttggtggagaacggcaaaggacccaatcagtcgattttgccgccgcctaaggatcttcccaagtgctaagtcctgcaactaaatagggcgacatatcaacatcacgccacgtccaagaagactaatgagtggtgatttaatgcgtggatttggctaacaaattgtatttttttgagcaaagcaaaaggttcggttttttttgctcatgtcaaaagtttgaataatggagaaaaaagagcattgtattataataatttgttttcattatattcaaattccattcatattttttattattgtctctaacacacctcttatatggtccaaagttgt

SEQ ID NO: 4 - protein, 558 aa, Arabidopsis thaliana
MGSHMVWFLFLVSFFSVFPAPSESMVRHYKFNVVMKNVTRLCSSKPTVTVNGRYPGPTIYAREDDTLLIKVVNHVKYNVSIHWHGVRQVRTGWADGPAYITQCPIQPGQVYTYNYTLTGQRGTLWWHAHILWLRATVYGALVILPKRGVPYPFPKPDNEKVIVLGEWWKSDTENIINEALKSGLAPNVSDSHMINGHPGPVRNCPSQGYKLSVENGKTYLLRLVNAALNEELFFKVAGHIFTVVEVDAVYVKPFKTDTVLIAPGQTTNVLLTASKSAGKYLVTASPFMDAPIAVDNVTATATVHYSGTLSSSPTILTLPPPQNATSIANNFTNSLRSLNSKKYPALVPTTIDHHLFFTVGLGLNACPTCKAGNGSRVVASINNVTFIMPKTALLPAHYFNTSGVFTTDFPKNPPHVFNYSGGSVTNMATETGTRLYKLPYNATVQLVLQDTGVIAPENHPVHLHGFNFFEVGRGLGNFNSTKDPKNFNLVDPVERNTIGVPSGGWVVIRFRADNPGVWFMHCHLEVHTTWGLKMAFLVENGKGPNQSILPPPKDLPKC

SEQ ID NO: 5 - protein, 587 aa, Zea mays
MAISSALPCSSLLMAAAQLMLLASVVVQVQGITRHYDFNVTMANVTRLCASKSIITVNGQFPGPKIVAREGDRLVIRVTNHAQHNISLHWHGIRQLRTGWADGPAYITQCPIQTGQSYVYNYTVVGQRGTLWWHAHISWLRATVYGPLVILPKLGVPYPFPAPYKEVPVIFGEWWLADTEVVIKQALQLGAGPNVSDAHTINGLPGPLYNCSAKDTYKLKVKPGKTYMLRLINAALNDELFFSVANHSLTVVEVDAVYVKPFTVDTLLIAPGQTTNVLLAAKPSYPGANYYMSAAPYSTARPATFDNTTVAGILEYELYPDAPRPSASAGSFNEALPLYRPTLPQLNDTSFVGNFTAKLRSLATPRYPAAVPRTVDRRFFFAVGLGTHPCPANATCQGPTNTTQFAASVNNVSFVLPTKALLHSHFTGLSSGVYSPDFPVAPLAPFNYTGTPPNNTNVASGTKLMVVPYGANVELVMQGTSILGVESHPLHLHGFNFFVVGQGYGNYDPVNDPSKFNLVDPVERNTVGVPAGGWVAIRFLADNPGVWFMHCHLEAHTTWGLRMAWLVLDGSLPHQKLLPPPSDLPKC

SEQ ID NO: 6 - protein, 582 aa, Zea mays
MATPYRLPCCCYALVTVLVLFFSVDATEGAIREYQFDVQMTNVTRLCSSKSIVTVNGQFPGPTVFAREGDFVVIRVVNHVPYNMSIHWHGIRQLRSGWADGPAYITQCPIQSGQSYVYKFTITGQRGTLWWHAHISWLRATVYGPIVILPKPGVPYPFPAPYDEVPVLFGEWWTADTEAVISQALQTGGGPNVSDAFTINGLPGPLYNCSAKDTFKLKVKPGKTYMLRIINAALNDELFFSIAGHPLTVVDVDAVYIKPITVETIIITPGQTTNVLLTTKPSYPGATYYMLAAPYSTARPGTFDNTTVAGILEYEDPTSSPPPHAAFDKNLPALKPTLPQINDTSFVANYTARLRSLATAEYPADVPREVHRRFFFTVGLGTHPCAVNGTCQGPTNSSRFAASVNNVSFVLPTTALLQSHFAGKSRGVYSSNFPAAPLVPFNYTGTPPNNTNVSNGTKLVVLPYGTSVELVMQGTSILGAESHPLHLHGFNFFVVGQGFGNFDPAKDPAKYNLVDPVERNTVGVPAAGWVAIRFRADNPGVWFMHCHLEVHVSWGLKMAWLVLDGDRPNEKLLPPPSDLPKC

SEQ ID NO: 7 - protein, 573 aa, Saccharum officinarum
MGAPPCLAFLLLFGTLLALLQSSHGTTRYYTFNVTMQNVTRLCTTRAIPTVNGKFPGPKIVTREGDRVVVKVVNNVKDNVTIHWHGVRQLRTGWSDGPAYVTQCPIQTGQSYVYNFTITGQRGTLLWHAHVSWMRATLYGPIVILPKRGVPYPFPVKPYKEVPIIFGEWFNADPEAIIAQALQTGAGPNGSDAFTINGLPGPLYNCSSKDTFRLKVLPGKWYLLRLINAALNDELFFSIANHTLTVVDVDAAYVKPFHTDIVLITPGQNTNVLLRAEPDAGCPAATHLMQARPYGTGQPGTFDNTTVAAVLEYAPAAGHIKSLPLFRPWLPALNDTAFAANYTARLRSLATPEYPASVPRGVDRSFFFAVGLGTNPCPANQTCQGPTNRTMFTASMNNVSFTMPTTALLQAHYDNIAGVYTTDFPVAPREPFNYTGTTPNNTNVSSGTKVVVLEYNTSVEVVLQGTSILGAESHPLHLHGFDFFVVGQGFGNYDSSKDPANFNLVDPVQRNTVGVPSAGWVAIRFFADNPGVWFMHCHLEVHTSWGLKMAWVVNDGPLPEQKLMPPPPDLPKC

SEQ ID NO: 8 - protein, 549 aa, Sorghum bicolor
VWQVQMTSVTRLCGTKGIVTVNGQYPGPTLFAREGDHVEVNVVNRSPYNISIHWHGVRQLLSGWADGPSYITQCPIQPGQSYVYRYQIVGQRGTLWWHAHISWLRATVYGPIVILPPAGVPYPFPAPDEEVPVMFGEWWRNDTEAVIAQALQTGGGPNVSDAYTINGLTGPLYNCSAQDTFKLKVKPGKTYMLRLINAALNDELFFSVANHTLTVVDVDALYVKPFTVDTLIIAPGQTSNVLLATKPTYPGAIYYMEARPYTNTQGTFDNTTVAGVLEYEDPSSSSSSGENQSNSNLPVFAPTLPPINDTSFVANYTAKLRSLASAEYPAAVPQDVDRRFFFTVGLGTHPCAGGVNGTCQGPNGSRFAASINNVSFVLPTTALLQAHFAGRSNGVYATNFPAFPPTPFNYAGTPPNNTNVMNGTKVAVLPFGTSVELVLQDTSILGAESHPLHLHGFNFFVVGQGFGNFDPTNDPAKFNLVDPVERNTVGVPAGGWVAIRFRADNPGVWFMHCHLEVHMSWGLKMAWLVLDGSLPNQKLPPPPLDLPQC

SEQ ID NO: 9 - protein, 585 aa, Sorghum bicolor
MAGGRRRLSPACLFLTVALVVLVALPELAAARTRRYTFNVTMATVTRLCVTKSIPTVNGRFPGPRIVVREGDRLVVQVHNNINNNVTFHWHGVRQLRSGWSDGPSFITQCPIRPGQSYAYDFRIVGQRGTLWWHAHFSWLRATLYGPLVILPPRGVPYPFPKPHAEVPLMLGEWFNADPEAVIKQALQTGGGPNVSDAYTFNGLPGPTYNCSGGGAGDTFKLRVKPGKTYLLRLVNAALNDELFFAVANHTLTVVQADASYVKPFTADTLVISPGQTMDVLLTAAAATAPSSTAAYAIAVAPYTNTVGTFDNTTAIAVLEYYGAPPQSAAALRNLPLPALPLYNDTGAVANFSAKFRSLASAQYPALVPRTVDRRFFFAVGLGADPCQSRVNGTCQGPNGTRFAATMNNVSFTMPKTSLLQAHYQRRYSGVLTANFPGVPPTPFNYTGTPPNNTFVTHGTRVVPLSFNTTVEVVLQDTSILGAESHPLHLHGYDFFVVGQGFGNYDAGNDTAKYNLVDPVQRNTISVPTAGWVAIRFVADNPGVWIMHCHLDVHLSWGLAMAWLVNDGPLPNQKLPPPPSDIPRC

SEQ ID NO: 10 - protein, 575 aa, Sorghum bicolor
MAAPSSLAFLLFFGTLLALPQSSHGATRYYTFNVTMQKVTRLCTTRAIPTVNGKFPGPKIVTREGDRVVVKVVNNVKDNVTIHWHGVRQLRTGWSDGPAYVTQCPIQTGQSFVYNFTITGQRGTLFWHAHVSWLRATLYGPIVILPKRGVPYPFLPVKPYKEVPIIFEWFNADPEAIIAQALQTGAGPNVSDAFTINGLPGPLYNCSSKDTFKLKVLPGKWYLLRLINAALNDELFFSIANHTLTVVDVDASYVKPFHTDIVLITPGQTTNVLLRAEPDAGCAAATHLMLARPYGTGQPGTFDNTTVAAVLEYAPAAAAGHIKSLPLFRPSLPALNDTAFAANYSARLRSLATPEFPATVPRAVDRSFFFAVGLGTNPCPANQTCQGPTNRTKFTASMNNVSFTMPTTALLQAHYDNIAGVYTADFPVAPLEPFNYTGTTPNNTNVSSGTKVVVLEYNTSVEVVLQGTSVLGAESHPLHLHGFDFFVVGQGFGNYDSSKDPAKFNLDDPVQRNTVGVPSGGWVAIRFFADNPGVWFMHCHLEVHTSWGLKMAWVVNDGPLPEQKLMPPPPDLPKC

SEQ ID NO: 11 - protein, 579 aa, Sorghum bicolor
MAISSALPCSLLMAALMLLASAVEVQGITRHYDFNVTMANVTRLCASKSIITVNGQFPGPKIVAREGDRLVIRVTNHAQHNISLHWHGIRQLRTGWADGPAYITQCPIQTGQSYVYNYTIVGQRGTLWWHAHISWLRATVYGPLVVLPKLGVPYPFPAPYKEVPVIFGEWWLADTEVVIQQALQLGAGPNVSDAHTINGLPGPLYNCSAKDTFKLKVKPGKTYMLRLINAALNDELFFSIANHSLTVVEVDAVYVKPFTVDTLVIAPGQTTNVLLAAKPFYPAANYYMTARTYSTGRPATFDNTTVAGILEYEYPDAPSAAASFNKALPLYRPALPQLNDTSFVGNFTAKLLSLATPEYPAAVPQTVDKRFFFTVGLGTHPCPANTTCQGPTNTTQFAASVNNVSFVLPTKALLHSHFAGLSSGVYESDFPVAPLAPFNYTGTPPNNTNVANGTKLMVIPYGTNVELVMQGTSILGIESHPLHLHGFNFFVVGQGYGNYDPVNDPPKFNLVDPVERNTVGVPAGGWVAIRFLADNPGVWFMHCHLEVHTTWGLRMAWLVLDGSLPHQKLLPPPSDLPKC

SEQ ID NO: 12 - protein, 8 aa, Artificial
  Consensus sequence
HWHGIRQL

SEQ ID NO: 13 - protein, 11 aa, Artificial
  Consensus sequence
INAALNDELFF

SEQ ID NO: 14 - protein, 23 aa, Artificial
  Consensus sequence
ESHPLHLHGFNFFVVGQGFGNFD

SEQ ID NO: 15 - protein, 8 aa, Artificial
  Consensus sequence
MHCHLEVH

SEQ ID NO: 16 - DNA, 21 bp, Artificial
  Primer
tcgaagagggtcaaagagttt

SEQ ID NO: 17 - DNA, 21 bp, Artificial
  Primer
tcttagccatgaaatgtgagc

SEQ ID NO: 18 - DNA, 21 bp, Artificial
  Primer
attgtgtaagcaaatcggcac

SEQ ID NO: 19 - DNA, 21 bp, Artificial
  Primer
tggcttgcttgagcataatct

SEQ ID NO: 20 - DNA, 21 bp, Artificial
  Primer
gcaagacaaaaacaatcgtga

SEQ ID NO: 21 - DNA, 21 bp, Artificial
  Primer
gaaatctgagggtggaggaag

SEQ ID NO: 22 - DNA, 21 bp, Artificial
  Primer
agtaatgaacagttgcggtgg

SEQ ID NO: 23 - DNA, 21 bp, Artificial
  Primer
tggtaactttggacgatcagg

SEQ ID NO: 24 - DNA, 21 bp, Artificial
  Primer
gttagaaactgtccatctcaa

SEQ ID NO: 25 - DNA, 21 bp, Artificial
  Primer
ctccacttgtgttgaagtaat

SEQ ID NO: 26 - DNA, 20 bp, Artificial
  Primer
atccggttgatgtgttgaga

SEQ ID NO: 27 - DNA, 20 bp, Artificial
  Primer
agagagatcggcttatgttg

SEQ ID NO: 28 - DNA, 19 bp, Artificial
  Primer
tatgccaaacaaacgagat

SEQ ID NO: 29 - DNA, 18 bp, Artificial
  Primer
ctgctggaggaggaggtc

SEQ ID NO: 30 - DNA, 20 bp, Artificial
  Primer
tgtaaagccggaaacttctc

SEQ ID NO: 31 - DNA, 20 bp, Artificial
  Primer
ttagggcctttaccattctc

SEQ ID NO: 32 - DNA, 18 bp, Artificial
  Primer
gagctattcttcgggatt

SEQ ID NO: 33 - DNA, 18 bp, Artificial
  Primer
gtctttaggcggtggtag

SEQ ID NO: 34 - DNA, 18 bp, Artificial
  Primer
gccgacgcatcttacctc

SEQ ID NO: 35 - DNA, 18 bp, Artificial
  Primer
ccaagaacgccatagcaa

SEQ ID NO: 36 - DNA, 21 bp, Artificial
  Primer
ttctcttgtgttcttcttctt

SEQ ID NO: 37 - DNA, 21 bp, Artificial
  Primer
gaacttctttgtgaggtttag

SEQ ID NO: 38 - protein, 8 aa, Artificial
  Consensus sequence
HWHGIRQL

SEQ ID NO: 39 - protein, 11 aa, Artificial
  Consensus sequence
INAALNDELFF

SEQ ID NO: 40 - protein, 23 aa, Artificial
  Consensus sequence
ESHPLHLHGFNFFVVGQGFGNFD

SEQ ID NO: 41 - protein, 7 aa, Artificial
  Consensus sequence
ADNPGVW

SEQ ID NO: 42 - protein, 587 aa, Zea mays; identical to SEQ ID NO: 5

SEQ ID NO: 43 - protein, 582 aa, Zea mays; identical to SEQ ID NO: 6

SEQ ID NO: 44 - protein, 584 aa, Zea mays
MAISSALPCSSLLMAAAQLKLLASVVVQVQGITRHYDFNVTMANVTRLCATKSIVTVNGQFPGPKIVAREGDRLIIRVTNQAQHNISLHWHGIRQLRTGWADGPAYITQCPIQTGQSYVYNYTVAGQRGTLWWHAHISWLRATVYGPLVVLPRPGVPYPFPAPYKEVPVIFGEWWLADTEVVVEQALQLGAGPNVSDAHTINGLPGPLYNCSAKDTYKLKVKPGKTYMLRLINAALNDELFFSVANHSLTVVEVDAVYVKPFTVDTLPIAPGQTTNVLLAAKPFYPGANYYMSAKPYSTARPATFDNTTVAGILEYEYPDAPSSSAASFDKALPLHRPTLPQLNDTSFVGNFTAKLRSLATPQYPAAVPRTVDRAFFFTVGLGTHPCPGNATCLGPTNTTQFAAAVNNVSFVLPTRALLHSHFAGLSSGVYSSDFPVAPLTPFNYTGTPPNNTNVASGTKLMVVPYGANVELVLQGTSILGVESHPLHLHGFNFFVVGQGYGNYDPVNDPPKFNLVDPVERNTVGVPAGGWVAIRFLADNPGVWFMHCHLEVHTTWGLRMAWLVLDGSLPHQKLLPPPSDLPKC

SEQ ID NO: 45 - protein, 576 aa, Zea mays
MGAAPPSSWLAFILFFGTLVALPQSSHGGGTTRHYTFNVTMKKVTRLCTTRAIPTVNGQFPGPKIVTREGDRVVVKVLNNVKDNVTIHWHGVRQLRTGWSDGPAYVTQCPIQTGQSFVYNFTITGQRGTLFWHAHVSWMRATLYGAIVILPKRGVPYPLPVKPYKDVPVIFGEWFNADPETIVAQALQTGAGPNVSDAFTINGLPGPLYNCSSKDTFKLKVLPGKWYLLRLINAALNDELFFSIANHTLTVVDVDAAYVKPFRTDIVLITPGQTTNVLLRAEPDAGCPAATHLMLARPYGTGQPGTFDNTTVAAVLEYAPPGHIRSLPLFRPSLPALNDTAFAANYSARLRSLATPDYPANVPRAVDRSFFFAVGLGTNPCPVNQTCQGPNGTMFTASMNNVSFNMPTTALLQAHYGSVAGVYTPDFPVAPLEPFNYTGTPPNNTNVSHGTKVVVLDYNTSVEVVLQATSILGAESHPLHLHGFDFFVVGQGFGNYDSSKDPPKFNLVDPVQRNTVGVPAGGWVAIRFFADNPGVWFMHCHLEVHTSWGLKMAWVVNDGPLPEQKLMPPPADLPKC

SEQ ID NO: 46 - protein, 588 aa, Zea mays
MGARRGLRRGQAAAAAFSACPFLALAVVLLALPELAAGDTHYYTFNVQMTNVTRLCVTKSIPTVNGEFPGPKLVVREGDRLVVKVHNHINYNVSFHWHGVRQLRNGWADGPSYITQCPIQGGQSYVYDFTVTGQRGTLWWHAHFSWLRVHLYGPLVILPKRGEGYPFPRPYKEVPILFGEWFNADTEAVINQALQTGAGPNVSDAYTFNGLPGPTYNCSSKDTYKLKVKAREGRTCSRLHQLRPQTNELFFGIANHTLTVVEADASYVKPFTVSTLVISPGQTMNVLLTTAPSPASPAYAMAIAPYTNTQGTFDNTTAAAVLEYAPTPPPVATRNNTLPPLPALPLYNDTGAVSNFSRNFRSLNSARYPARVPAAVDRHLLFTVGLGTDPCPYTNQTCQGPNGTKFAASVNNNSFFRPRTALLEAHYRRRYAGVLLGDFPTAPPHPFNYTGTPPNNTFVQHGTRVVPLRFNASVELVLQGTSIQGAESHPLHLHGYNFFVVGQGFGNFDPVNDPPGYNLADPVERNTISVPTAGWVAVRFLADNPGVWLMHCHFDVHLSWGLSMAWLVNDGPLPNEKMLPPPSDLPKC

SEQ ID NO: 47 - protein, 582 aa, Zea mays
MAVGRRLSPACLLLLRLTVALVVLTALPELAAARTRRYTFNVTMATVTRLCVTKSVPTVNGQFPGPRLVVREGDRLVVQVHNNINSNVTFHWHGVRQLRSGWADGPSYITQCPIRPGQSYAYDFRIVGQRGTLWWHAHFSWLRATLYGPLVILPPRGVPYPFPKPDRQVTLMLGEWFNADPEAVIKQALQTGGAPNVSDAYTFNGLPGPTYNCSSGDDTFRLRVRPGRTYLLRLVNAALNDELFFAVANHTLTVVQADASYVKPFAAATLVISPGQTMDVLLTASASAAPSSAFAIAVAPYTNTVGTFDNTTAVAVVEYGPHQSAAALRSLPLPALPRYNDTAAVANFSAMFRSLASARYPARVPRTVDRRFFFTVGLGADPCRSRVNGTCQGPNGTRFAASMNNVSFAMPRTTSLLQAHYQRRYSGVLAANFPAVPPTRFDYTGAPPNNTFVTHGTRVVPLSFNTTVEVVLQDTSVLGAESHPLHLHGYDFFVVGTGFGNYDATNDTARYNLVDPVQRNTVSVPTAGWVAIRFVADNPGVWIMHCHLDVHLTWGLAMAWLVNDGPLPNQKLPPPPSDIPRC

SEQ ID NO: 48 - protein, 572 aa, Brachypodium distachyon
MGAKCLSLLVFLGTSLLLPQLLLAAMTRYYTFNVTMKKVTRLCNTRAIPTVNGKFPGPKIVTREGDRVVVKVVNNVKHNVTIHWHGVRQLRTGWSDGPAYITQCPIQTGQSYVYNFTVTGQRGTLFWHAHVSWMRATLYGPIVILPKLGVPYPFPKPFKDVPIMFGEWFNVDPEAIIAQALQTGGGPNVSDAYTINGLPGPLYNCSSRDTFKLKVQPGKWYLLRLINAALNDELFFSIANHTLTIVDVDASYVKPFDTDVVLVTPGQTTNVLLHAKPDEGCQPATHLMLARPYATSRPGTYDNTTVAAVLEYSPSGQIKSRPLFRPTLPVFNDTSFAANYSAKHRSLASSEYPANVPRRIDRPFFFAVGLGTTPCPTHQGCNGPTNDTKFSASMNNVSFNMPTTALLKAHYDGNTAGVYTADFPAMPTQPFNYTGTPPNNTNVSNGTKVAVLPYNASVEVVLQDTSIQGAESHPLHLHGFDFFVVGQGVGNYNASMHPAGFNLLDPVQRNTVGVPAGGWVAIRFYADNPGVWFMHCHLEVHTSWGLKMAWVVNDGPLPDQKLMPPPSDLPKC

SEQ ID NO: 49 - protein, 577 aa, Brachypodium distachyon
MAMAISSGLPACSVVMATLMVLIIQAQGITRHYDFDVQMAKVTRLCGSKSIVTVNGQFPGPELVAREGDRVHVRVTNHVSHNMSLHWHGIRQMQTGWADGPAYITQCPIQMGQTYVYKFTITGQRGTLWWHAHISWHRATVYGAIVILPKLGVPYPFAAPHKEVPVIFGEWWAADTEVVMSQALKVGGAPNISDAFTINGLPGPLYNCSAQDTFKLKVTPGKTYLLRLINAALNDELFFSVANHTLTVVEVDAVYVKPFTVKTIVISPGQTTNVLLTAKPVNPKANFYMSAAPYSVIRPGTFDNTTVAGILEYHEDPSSSSSFDKNLPLFKPMLPRFNDTKFVTNFTTKLRSLATTKYPAAVPQTVDKRFFFTIGLGTLPCPKNMTCQGPNGTQFAAAVNNVSLVLPTKALLQSHFTGLTTGVYASDFPAMPLSPFNYTGTPPNNTNVATGTKLLALPFNTSVELVMQDTSVLGIESHPLHLHGFNYFVVGQGFGNYDSAKDPAKFNLVDPVERNTVGVPAGGWVAIRFLADNPGVWFMHCHLEVHTTWGLRMAWLVHDGSKPNQKLLPPPSDMPKC

SEQ ID NO: 50 - protein, 599 aa, Brachypodium distachyon
MAMAIPSGVPSPCSLLMAILVLLIVQVQGITRHYDFNVQMANVTRLCATKSIMTVNGEFPGPALVAREGDRVLVRVTNQVSHNMTLHWHGIRQLRSGWADGPAYVAQCPIQIGQSYVYNFNITGQRGTLWWHAHISWIRATVYGAIVILPELGIPYPLAAPHEEVPILFGEWWKANTEAVVKQALQTGGAPNISDAFTINGLPGPLYNCSAKGMNELVLKVEAGKTYLLRLINAALNDELFFVIANHKLTVVEVDAVCVKPFTVNTLVISPGQTTNVLLTAKPFNPKANFYMSAVPYSTIRPGTFDNTTVTGILEYHNPNSGSASSSFDKDLPLFKPTMPRFNDTGPVTNFTTKLRSLATATYPVIVPRSVDKRFLFTIGPGTLPCPVNMTCQGPTNVTRFAAAMNNVSLVFPSTALLQSHYTGMGMSKGVYASNFPTAPLTPFSYTGTPPNNINVAKGTRLLVLPFNTSVELVMQETSILGVESHPLHLHGFNFFVVGQGFGNYDAVNDLAKFNLVDPVERNTVGVPASGWVAIRFLADNPGVWFMHCHLEVHTTWGLRMAWLVLDGSLANQKLNRHLIFPNASRHTLPEFVLPRLIG

SEQ ID NO: 51 - protein, 581 aa, Brachypodium distachyon
MAASPGLPAPWSLFMATLVLLIVQAQGITRHFDFNVQMANVTRLCATKSIVTVNGEFPGPALVAREGDRVLVRVTNQVSHNMTLHWHGIRQLRSGWADGPAYVAQCPIQTGQSYVYNFTITGQRGTLWWHAHISWLRATVYGAIVILPEFGVPYPFAAPHEEVPILFGEWWKADTEAVVKQALQTGGAPNISDAFTINGLPGPLYNCSAKDTFKLKVQPGKTYLLRLINAALNDELFFSIAKHRLTVVEVDAVYVKPFTVDTLVISPGQTTNVLLTAKLFYPKANFFMSAVPYSNIRPGTFDNTTVAGILEYHNPSSGSVSSSFNKDLPLFRPKLPRFNDTGLVTKFTAKLRSLATASYPAAVPQSVDKRFFFTIGLGTLPCPTNTTCQGPTNITRFAAAVNNVSLVLPSTALLQSHYTGMGMSKGVYASNFPTAPLSTFNYTGTPPNNTNVAQGTRLLVLPFNASVELVMQDTSILGIESHPLHLHGFNFFVVGQGFGNYDVVNDPAQFNLVDPVERNTVAVPAGGWVAIRFIADNPGVWFMHCHLEVHTTWGLRMAWLVRDGSLPNQKLLPPPSDLPKC

SEQ ID NO: 52 - protein, 576 aa, Brachypodium distachyon
MGALRGLRRHAAASACPFLAFAVLLALPGLAAGITRHYTFDVQMTNVTRLCATKSIPTVNGQFPGPRLVAREGDRLVVKVHNHINYNVSFHWHGIRQLRNGWADGPAYITQCPIQGGQSYVYDFTITGQRGTLWWHAHFSWLRVHLYGPLVILPKRAEGYPFPLPYKEFPIMFGEWFKADSEAVINQALQTGAGPNVSDAYTFNGLSGPTYNCSSKDTYKLKVQPGRTYMLRLINSALNNELFFGIANHTLTVVEADANYVKPFTSKTVVISPGQTMNVLLTTSSNPASRAFAMSIAPYTNTQGTFDNTTATAVLEYASTRPSSTQNLAMPALPRYNDTNAVANFSSNFRSLASAQYPARVPQAVDRHVLFTVGLGTDPCPSNQTCQGPNGTKFAASINNNSFVRPKTALLEAHYQSRYAGVLMANFPTTPPHPFNYTGTPPNKTFVAHGTRVVPLSFNTTVELVMQGTSIQGAESHPLHMHGFNFFVVGQGFGNYDPVNDPAKYNLIDPVERNTVSVPTAGWVAVRFLADNPGVWLMHCHFDVHLSWGLSMAWLVNDGPLPNQKMLPPPSDLPKC

SEQ ID NO: 53 - protein, 580 aa, Brachypodium distachyon
MGGAHHGLLRCASPACLHLAFFLLLAVEPGLTAGLTRRYTFNVTMATVTRLCLTKSIPTVNGQFPGPKISVREGDRLVVNVHNNINNNVTFHWHGVRQLRSGWADGPAYITQCPIRPGQSYVYSFRIVGQRGTLWWHAHFSWLRATLHGPMVILPPLGVPYPFPKPYREVPLMLGEWFNADPEAVIKQALQTGGGPNVSDAYTFNGFPGPTYNCSAKSTYKLKVKPGRTYMLRLINAALNDELFFAVANHTLTVVQADASYVKPFTANTLVISPGQTMDVLLTAAASTSSSAFAIAVAPYTNTVGTFDNTTAIAVLEYSPQRPSMLRNLPAPSLPLYNDTNAVTNFSSKFRSLDNARYPAQVPMTVDRHFFFTVGLGADPCQSPINGTCQGPNNTRFAASINNVSFIMPKTSLLQAHYQRMYKGVLMANFPTAPVRKFNYTGTPPNNTFVTHGTRVVPLAFNTTVEVVLQDTSIQGAESHPLHLHGYDFYVVGTGFGNYDANNDTAKYNLVDPVKRNTISVPTAGWVAIRFIADNPGVWIMHCHLDVHLSWGLSMAWLVNDGPLPNQKLPPPPADIPKCS

SEQ ID NO: 54 - protein, 577 aa, Brachypodium distachyon
MAVSSSLLCLLFAALTATLVGAQAVTRKYQFDVQMTSVTRLCSTKSIVTVNGQYPGPTLFAREGDHVEVNVVNNSPYNMTIHWHGVRQLRSGWYDGPAYITQCPIQPGQSYVYRFTITGQRGTLWWHAHVSWLRATVHGPIVILPPLGVPYPFSPVPYKEVPLMFGEWWKNDTEAVIAQALQTGGGPNISDAFTMNGLPGPLYNVCSSAKGETFKLKVEPGKTYMLRLINAALNDELFFAVANHTLTVVDVDALYVKPFAVESLVIAPGQTSNVLLTANNNPNARYYMLARPYTNTQGTFDNTTVAGILEYEDTSNGKAPSTSSLPILRPTLPEINDTSVVSNYTAKLRSLASAEYPASVPQQVDREFFFTVGLGTHPCQSLINGTCQGPNNTRFAASINNISFVLPTTALLQSHYTGMSNGVYASNFPFYPLRPWNYTGTPPNNTNVMNGTKALVLPFGVAVELVMQDTSILGAESHPLHLHGFNFFVVGQGFGNYNPSSDPAKFNLVDPVERNTVGVPAGGWVAIRFRADNPGVWFMHCHLEVHMSWGLKMAWVVLDGARPDQKLPPPPADLPKC

SEQ ID NO: 55 - protein, 578 aa, Brachypodium distachyon
MAATFRSSSLLVVAALMVLSINLAEGDTVEYQFDVETMNVTRLCSSKSIVAVNGQFPGPTVLAREGDLVVVRVVNKAQYNMSIHWHGVRQLRSGWADGPAYITQCPIQPGQSYVYKFTISGQQGTLWWHAHISWLRATVYGPIVILPKLGVPYPFPAPFKEVPLMFGEWWKADTEAVISQALQTGGGPNVSDAFTINGLPGPLYNCSAKDTFKLKVKPGKMYMLRIINSALNDELFFSIAGHLLTVVDVDAVYVKPVTVETLLITPGQTTNVLLTTKPSYPGANYYMLASPYSTAASGTFDNTTVAGILEYEHDEYPGSSASFNKNLPLFKPTMPQINDTSFVSNFTAKLRSLANEDYPADVPREVDRRFFFTVGLGTHPCAGANGTCQGPNGSRFAAAVNNVSFVLPTTALLQSHFTGMSNGVYESNFPAMPSSPFNYTGTPPNNTNVSNGTKLVVLSYGESVELVMQGTSILGAESHPFHLHGFNFFVVGQGFGNFDPMSDPAKYNLVDPVERNTVGVPAAGWVALRFRADNPGVWFMHCHLEVHVSWGLKMAWLVQDGSLSNQKLLPPPSDLPKC

SEQ ID NO: 56 - protein, 578 aa, Oryza sativa
MGARCLALLLLYGTLLLLLLLPQLPLAGAATRYYTFNVKLQNVTRLCNTRAIPTVNGKFPGPKIVTREGDRVVVKVVNNIKDNITIHWHGVRQMRTGWSDGPAYVTQCPIQTGQSYVYNFTINGQRGTLFWHAHVSWLRSTLYGPIIILPKAGLPLPFTEPHKDVPIIFGEWFNADPEAIVAQALQTGGGPNVSDAYTINGLPGPLYNCSSKDTFRLKVQPGKMYLLRLINAALNDELFFSVANHTLTVVDVDASYVKPFDTDVVLITPGQTTNVLLRAKPTAEAAGATHLMMARPYATGRPGTYDNTTVAAVLEYAPPGHIKSLPLLRPSLPALNDTAFAAGFAAKLRSLACPDYPSNVPRRVDKPFFFAVGLGTTPCPGSNNQTCQGPTNTTKFTASINNVSFDMPTTALLQAHYTGQSAGVYTADFPASPLEPFNYTGTPPNNTNVSNGTRVVVLPYNASVEVVLQDTSILGAESHPLHLHGFDFFVVGQGTGNYDPSKHPAEFNLVDPVQRNTVGVPAGGWVAIRFFADNPGVWFMHCHLEVHTTWGLKMAWVVNDGPLPEQKLMPPPSDLPMC

SEQ ID NO: 57 - protein, 577 aa, Oryza sativa
MAISYLLRSSILAVAALLLFSVNLAKGDIREYQFDVKTTNVTRLCSSKSIVTVNGQFPGPTVFAREGDLVVIRVINHSPYNMSIHWHGIRQLRSGWADGPAYITQCPIQPGGSYVYKYTITGQRGTLWWHAHISWLRATVYGPIIILPKAGVPYPFPAPDKEVPVVFGEWWKADTEAVISQATQTGGGPNVSDAFTINGLPGPLYNCSAKDTFKLKVEAGKTYMLRLINAALNDELFFSIAGHTLTVVDVDAVYVKPFTVDTLLITPGQTTNVLLTTKPSYPGATFYMLAAPYSTAMSGTFDNTTVAGILEYEDPSSHSTAAFNKNLPVLRPTLPQINDTSFVSNYTAKLRSFATAEYPANVPQQVDTRFFFTVGLGTHPCAVNGTCQGPNGSRFAAAVNNVSFVLPSTALLQSHYTGRSNGVYASNFPAMPLSPFNYTGTPPNNTNVSNGTRLVVLPYGASVELVMQGTSVLGAESHPFHLHGFNFFVVGQGFGNFDPVNDPAKYNLVDPVERNTVGVPAAGWVAIRFLVDNPGVWFMHCHLEVHVSWGLKMAWVVQDGSLPNQKILPPPSDLPKC

SEQ ID NO: 58 - protein, 574 aa, Oryza sativa
MAAASSVLRCCLLVAALMTLSAMGAEAITRQYLFDVQTTSVTRLCSTKSIVTVNGQYPGPTLFAREGDHVEVTVVNHSPYNMSIHWHGIRQLLSGWADGPSYITQCPIQPGGSYVYRFTITGQRGTLWWHAHISWLRATVHGPMVILPPAGVGYPFPAPHEEVPIMFGEWWNNDTEAVISQALQTGGGPNISDAYTLNGLPGPLYNCSAQDTFKLKVKPGKTYMLRLINAALNDELFFSIANHTLTVVDVDALYVKPFTVDTLIIAPGQTSNVLLTAKPTYPGASYYMLARPYTTTQGTFDNTTVAGVLEYDDPCPTTAAGKIVPIFSPTLPQINDTNAVSNFTAKLRSLASAGYPAAVPQQVDHRFFFTVGLGTHPCAVNGTCQGPNGSRFAASINNVSFVLPATALLQSHFAGKSKGVYASNFPYYPLNPFNYTGTPPNNTNVMNGTKVLVLPYGANVELVMQDTSILGAESHPLHLHGFNFFVVGQGFGNFDPINDPAKFNLYDPVERNTVGVPAGGWVAIRFHADNPGVWFMHCHLEVHMSWGLKMAWLVLDGSRPDQKLPPPPLDLPKC

SEQ ID NO: 59 - protein, 549 aa, Oryza sativa
MAAASSVLRCCLLVAALMTLSAMGAEAITRQYLFDVQTTSVTRLCSTKSIVTVNGQYPGPTLFAREGDHVEVTVVNHSPYNMSIHWHGIRQLLSGWADGPSYITQCPIQPGGSYVYRFTITGQRGTLWWHAHISWLRATVHGPMVILPPAGVGYPFPAPHEEVPIMFGEWWNNDTEAVISQALQTGGGPNISDAYTLNGLPGPLYNCSAQDTFKLKVKPGKTYMLRLINAALNDELFFSIANHTLTVVDVDALYVKPFTVDTLIIAPGQTSNVLLTAKPTYPGASYYMLARPYTTTQGTFDNTTVAGVLEYDDPCPTTAAGKIVPIFSPTLPQINDTNAVSNFTAKLRSLASAGYPAAVPQQVDHRFFFTVGLGTHPCAVNGTCQGPNGSRFAASINNVSFVLPATALLQSHFAGKSKGVYASNFPYYPLNPFNYTGTPPNNTNVMNGTKVLVLPYGANVELVMQDTSILGAESHPLHLHGFNFFVVGQGFGNFDPINDPAKFNLYDPVERNTVGVPAGGWVAIRFHADNPGMYSEPHRFPCDSTFAPT

SEQ ID NO: 60 - protein, 579 aa, Oryza sativa
MTMAISSALPSPLLLAASLLLLIVQAQGITRHYEFNVQMANATRLCNTKSMVTVNGQCPGPELVAREGDRVVIRVTNNVAHNISLHWHGVRQVRTGWADGPAYITQCPIQTGQSYVYNFTVAGQRGTLWWHAHISWLRATVYGALVILPKLGVPYPFPAPHKEVPVIFGEWWNADTEEVVNQAVQTGGGPNVSDAFTINGLPGPLYNCSAQDTFKLKVKPGKTYMLRLINAALNEELFFAVANHTLTVVEVDAVYVKPFTVDTLVISPGQTTNVLLTAKPYYPGANFYMSAAPYSTARPGTFGNTTVAGILEYENPAMSPSAASFVKGLPLFKPTLPQLNDTDFVTNFTDKLRSLATPEYPAAVPQSVDKRFFFTVGLGTLPCPANMTCQGPNNTQMAASMNNVSFVLPARALLQSHFTGLSSGVYAPDFPVAPLSPFNYTGTPPNNTNVKTGTKLLVLRYNTSVELVMQDTSILGIESHPLHLHGFNFFVIGQGFGNYDAVNDPAKFNLVDPVERNTVGVPAGGWVAIRFLADNPGVWFMHCHLEAHTTWGLRMAWLVLDGSHPNQKLLPPPSDLPKC

SEQ ID NO: 61 - protein, 513 aa, Oryza sativa
MATVTRLCVTKSVPTVNGQFPGPKLVVREGDTLVIRVTNNINNNVTFHWHGIRQVRSGWADGPAYITQCPIRSGGSYVYRFTVTGQRGTLWWHAHFSWLRATLYGPLVILPPRGVAYPFPKPHREVPLLLGEWFNADPEAVIKQALQTGGGPNVSDAYTFNGLPGPTYNCSSSNDTFKLRVRPGKTYLLRLINAALNDELFFGVANHTLMVVQADASYVKPFAATALVISPGQTMDVLLTAAANNPPSRSFAIAVAPYTNTVGTFDNTTAVAVLEYYGAATSAAALRSLPLPSLPAYNDTGAVANFSASFRSLASAQYPARVPRTVDRHFFFAVGLGADPCQSPVNGTCQGPNNTRFAASMNNVSFVMPRTSLLQAHYQRRYNGVLAANFPAAPRTPFNYTGTPPNNTFVTHGTRVVPLSFNTTVEVVLQDTSILGAESHPLHLHGYDFYVVGTGFGNYDASNDTAKYNLVDPVQRNTISVPTAGWVAIRFVADNPGWLPALYLYLHLKREFL

SEQ ID NO: 62 - protein, 547 aa, Oryza sativa
MGTPRGLRNAGSSSSACRFLAAFAVLLALPTLTAGLTRHYTFNVQMTNVTRLCVTKSIPTVNGQFPGPKLVVREGDRLVVKVHNHMNYNVSFHWHGILQLRNGWADGPSYITQCPIQGGGSYVYDFTVTGQRGTLWWHAHFSWLRVHLYGPLVILPKRGEGFPFPRPYKELPPIMFGEWFNADTEAVINQALQTGAGPNISDAYTFNGLPGPTYNCSSKDTYKVKVQPGRTYLLRLINSALNDELFFGIANHTLTVVEADANYVKPFTAKTLVISPGQTMNLLLTTAPNPGSPVYAMAIAPYTNTQGTFDNTTAVAVLEYAPTRASATGNNNLPLPPLPRYNDTNAVANFSSKFRSLATARYPARVPRAVDRHVLFTVGLGTDPCPSNQTCQGPNGTKFAASINNNSFVRPRVALLEAHCQRRVVPLAFNTSVELVLQGTSIQGAESHPLHMHGFNFFVVGQGFGNYDPVNDPANYNLVDPVERNTVSVPTGGWVAVRFLADNPGVWLMHCHFDVHLSWGLSMAWLVNDGPLPSQKMLPPPSDLPKC

SEQ ID NO: 63 - protein, 581 aa, Populus sp.
MGVSFLPSPAFLGLLLFSFVTLSLHPKPAVATTRHYKLDVMLQNVTRLCHTKSMVTVNGKFPGPRIVAREGDRLLIKVVNHVQNNISIHWHGIRQLRSGWADGPAYVTQCPIQTGQSYVYNYTIVGQRGTLWWHAHISWLRSTLYGPLIILPKLGTPYPFVKPYKEVPIIFGEWFNADPEAIINQALQTGGGPNVSDAYTINGLPGPLYNCSAKDTFKLKVKPGKTYLLRLINAALNDELFFSIANHTFTVVEADAVYVKPFDTKTLLIAPGQTTNVLLKTKPHHPNAKFFMTARPYVTGQGTFDNSTVAGILEYEESHKTIQSSHSTKRLPLFKPNLPPLNDTSFATKFTSKLRSLANAQFPANVPQKVDRQFFFTVGLGTHSCPQNQTCQGPNGTMFAASVNNVSFAMPTTALLQAHHFGQSNGVYTPDFPINPLTPFNYTGNPPNNTMVSNGTKLVVLPFNTTVELIMQDTSILGAESHPLHLHGFNFFVVGQGFGNFDPNKDPANFNLIDPIERNTVGVPSGGWVAIRFLADNPGVWFMHCHLEVHTSWGLKMAWVVLDGKLPNQKLLPPPADLPRC

SEQ ID NO: 64 - protein, 581 aa, Populus sp.
MGVYLLPSPASLAVFLSSFVTLFVHPRPAIAITRHYKFDVMLQNVTRLCHTKSMVTVNGKFPGPCIVAREGDRLLIKVVNHVQNNISIHWHGIRQLRSGWADGPAYVTQCPIQTGQSYVYNYTIVGQRGTLWWHAHISWLRSTLYGPLIILPKLGTPYPFAKPDKEVPIIFGEWFNADPEAIINQAMQTGGGPNVSDAYTINGLPGPLYNCSAKNTFKLKVKPGKTYLLRLINAALNEELFFSIANHTLTVVGVDAIYVKPFDTETLLIASGQTTDVLLKTKPHHPDAKFFMSARPYVTGQGTFDNSTVAGILEYEVARKTIQSSHTSKRLPLYKPNLPPLNDTSFATNFTSKLRSLASAEFPANVPQKVDRHFFFTVGLGTNPCSKNQTCQGPNGTRFAASVNNVSFVMPTKALLEAHHFGQSKGVYSPNFPISPLIPFDYTGTPQNNTMVSHGTKLVMLPFNTSVELIMQDTSILGAESHPLHLHGFNFFVVGQGFGNFDPKKDPANFNLVDPVERNTVGVPSGGWVAIRFLADNPGVWFLHCHVELHMSWGLMMAWVVLDGKLPNHRLLPPPVDLPKC

SEQ ID NO: 65 - protein, 581 aa, Populus sp.
MGASFLPSPAFLAVFLISFVTLSIHPEPALAITRHYKFDVMLQNVTRLCHTKSIVTVNGKFPGPRIVAREGDRLLIKVVNHVQNNISIHWHGIRQLRSGWADGPAYITQCPIQTGQSYVYNYTIVGQRGTLWWHAHISWLRSTLYGPLIILPKLGTTYPFAKPHKEVPIIFGEWFNADPEAIINQAMQTGGGPNVSDAYTINGFPGPLYNCSAKDTFKLKVKPGKTYLLRMINAALNDELFFSIANHTLTVVDVDAIYVKPFDTETLLIAPGQTTNVLLKTKPHHPNASFFMSARPYVTGQGTFDNSTVAGILEYEESNKTIKSSHSPKKLPFYKPNLPPLNDTSFATNFTSKLRSLASAEFPANVPQKVDRQFFFSVSLGTNPCSKNKTCQGPNGTMFAASVNNVSFVMPTKALLQAHHFGQSKGVYSPNFPINPLIPFNYTGTPPNNTMVSNGTKLVVLPFNTSVELIMQDTSILGAESHPLHLHGFNFFVVGEGFGNFDPKKDPANFNLVDPVERNTVGVPSGGWVAIRFLADNPGVWFMHCHLEVHTSWGLKMAWVVLDGKLPNQKLLPPPADLPKC

SEQ ID NO: 66 - protein, 581 aa, Populus sp.; identical to SEQ ID NO: 65

SEQ ID NO: 67 - protein, 581 aa, Populus sp.
MGASLLPPPAFLAVFLFSFVTLSVNPEPALAITRHYKFDVMLQNVTRLCHTKSMVTVNGKFPGPRIVAREGDRLVITVVNHVQNNISIHWHGIRQLRSGWADGPAYVTQCPIQTGQSYVYNYTIVGQRGTLWWHAHISWLRSTLYGPIILLPKLGTPYPFAKPYKEVPIIFGEWFNADPEAIINQAMQTGGGPNVSDAYTINGLPGPLYNCSAKDTFKLKVKPGKTYLLRMINAALNDELFFSIANHTVTVVDVDAVYVKPFDAETLLITPGQTTNVLLKTKPDYPNAQFFMSARPYATGQGTFDNSTVAGILEYEVPNKTSQSNHSTKKLPLYKPNLPPLNDTSFATNFSSKLRSLASADFPANVPQKVDRQFVFTVGLGTNPCSKNQTCQGPNGTRFAASVNNVSFVMPSTALLQAHHFGQSRGVYSPYFAISPLIPFNYTGTPPNNTMVSNGTKLVVLPFNTSVELIMQDTSILGAESHPLHLHGFNFFVVGQGFGNFDPSKDPANFNLVDPVERNTVGVPSGGWVAIRFLADNPGVWFMHCHLEVHTSWGLKMAWVVLDGKLPNQKLLPPPADLPKC

SEQ ID NO: 68 - protein, 581 aa, Populus sp.
MGASLLPPPAFLAVFLFSFVTLSVNPEPALAITRHYKFDVMLQNVTRLCHTRSMVTVNGKFPGPRIVAREGDRLVIRMVNHVQNNISIHWHGIRQLRSGWADGPAYVTQCPIQTGQSYVYNYTIVGQRGTLWWHAHISWLRSTLHGPIILLPKLGTPYPFAKPYKEVPIIFGEWFNADPEAIISQAMQTGGGPNVSDAYTINGLPGPLYNCSAKDTFKLKVKPGKTYLLRMINAALNDELFFSIANHTVTVVDVDAVYVKPFDAETLLITPGQTTNVLLKTKPDYPNAQFFMSARPYATGQGTFDNSTVAGILEYEVPNKTSQSNHSTKKLPLYKPNLPPLNDTSFATNFSSKLRSLASADFPANVPQKVDRQFFFTVGLGTNPCSKNQTCQGPNGTRFAASVNNVSFVMPTTALLQAHHFGQSRGVYSPYFPISPLIPFNYTGTPPNNTMVSNGTKLVVLPFNTSVELIMQGTSILGAESHPLHLHGFNFFVVGQGFGNFDPSKDPANFNLVDPVERNTVGVPSGGWVAIRFLADNPGVWFMHCHLEVHTSWGLKMAWVVLDGKLPNQKLLPPPADLPKC

SEQ ID NO: 69 - protein, 561 aa, Brachypodium distachyon
MPRHLSQLLLVVMVNCVLLQALSVHAITRHYKFNVVMRKMSRLCSTKTILTVNGKFPGPTLYAREGDNVLVKVVNHVPHNVTIHWHGVRQIRTGWYDGPAYITQCPIQPGSSFLYNFTITGQRGTLLWHAHINWLRATVHGAIVILPKLGVPYPFPAPHKEAVVVLGEWWKADIETIINRAMQLGVGPNISDSHTINGHPGPMSDCASSQDGFKLNVESGKTYMLRIINAALNDDLFFKIAGHKLTVVEVDAVYTKPYKTDILLITPGQTTNVLLAADQSAGRYLLSISPFMDAPVQVDNTTGTAILHYTNTVSAAARLTLFKPPPQNATLIASKFADSLRSLNSKEYPANVPRTVDHSLFFTIGVGVNPCPNCINGTRVVGTINNLTFVMPSTPILQAYYYNIPGVFTEDFPATPPHKFNYTGSGPKNLRTMNGTRVYRLPYNASVQVILQDTGIISTESHPIHLHGFNFFVVGRGIGNYSPKTSPSTFNLIDPIERNTIGVPTGGWTAIRFRADNPGVWFMHCHFEVHTSWGLKMVFVVENGKRPSETLIPPPKDLLQC

SEQ ID NO: 70 - protein, 564 aa, Oryza sativa
MAVLPESRRLSLLLMAACFLLQALSAHAITRHYKFNVVMRNMTRLCSTKPILTVNGKFPGPTLYAREGDNVLVKVVNHVAHNVTIHWHGVRQIRTGWYDGPAYITQCPIQPGSSFLYNFTITGQRGTLLWHAHINWLRATVHGAIVILPKLGVPYPFPAPHKEAVIVLGEWWKEDTETVINQAMQLGVGPNISDSHTINGHPGPLSECASSQDGFKLSVENGKTYMLRIINAALNDDLFFKVAGHELTVVEVDAVYTKPFKTDTLLITPGQTTNVLVRANQGAGRYLLSVSPFMDAPVQVDNKTGTATLHYANTVSSSMASLTLVKPPPQNATHIVSKFTDSLHSLNSKEYPANVPQTVDHSLLLTVGVGVNPCPSCINGTRVVGTINNVTFIMPSTPILQAHYYNIPGVFTEDFPATPLHKFNYTGSGPKNLQTMNGTRVYRLPYNASVQVVLQDTGIISPESHPIHLHGFNFFVVGKGVGNYNPRTSPSTFNLIDPIERNTIGVPTGGWTAIRFRSDNPGVWFMHCHFEVHTSWGLKMAFVVDNGKRPSETLIPPPKDLPQC

SEQ ID NO: 71 - protein, 558 aa, Populus sp.
MEYACWLRFMLLAVCLFPALVQCRVRHYKFNVVMKNTTRLCSRKPIVTVNGRFPGPTLYAREDDTVLVKVVNHVKYNVSIHWHGIRQLRTGWADGPAYITQCPIQPGQSYVYNFTITGQRGTLLWHAHILWLRATVHGALVVLPKRGIPYPFPAPHKEVLVVLAEWWKSDTEAVINEALKSGLAPNVSDAHTINGHPGAVSTCSSQGGFTLPVKSGETYMLRLINAALNEELFFKIAGHKLTVVEVDATYVKPFKTDTVLIAPGQTTNVLVTTNKNTGKYLVAASPFMDSPIAVDNMTATATLQYSGALANSPTTLTTPPPKNATAVANQFTNSLRSLNSRRFPAKVPLNVDHNLFFTVSLGVNPCPSCKAGNGSRVVASINNVTFVMPTTALLQAHFLNISGVFTTDFPAKPPHVFNYTGTPPTNLQTKSGTKVYRLSYNSTVQLVMQDTGIISPENHPIHLHGFNFFAVGRGVGNYNPKTDTKKFNLVDPVERNTIGVPSGGWVAIRFRADNPGVWFMHCHLEVHTTWGLKMAFLVDNGKGPKESLLPPPSDLPKC

SEQ ID NO: 72 - protein, 559 aa, Populus sp.
MEYSNWLIRFMLLAVCLLPALVECRIRHYKFNVVMKNTTRLCSRKPIVTVNGRFPGPTLYAREHDTVLVKVVNHVKYNVSIHWHGIRQLRTGWADGPAYITQCPIQPGQSYVYNFTITGQRGTLLWHAHILWLRATVHGALVVLPKRGIPYPFPAPHKEVVVVLAEWWKSDTEAVINEALKSGLAPNVSDAHTINGHPGAVSACSSQGGFTLPVKSGETYMLRLINAALNEELFFKIAGHKLTVVEVDATYVKPFKTDTVLIAPGQTTNVLVTTNKNTGKYLVAASPFMDAPIAVDNMTATATLQYSGALANSPTTLTTPPPKNATAVANQFTNSLRSLNSRRFPAKVPLNVDHNLFFTVGLGVNPCPSCKAGNGSRVVASINNVTFVMPTTALLQAHFLNISGVFTTDFPAKPPHVFNYTGTPPTNLQTKSGTKVYRLSYNSTVQLVMQDTGIISPENHPIHLHGFNFFAVGRGVGNYNPKTDTKKFNLVDPVERNTIGVPSGGWVAIRFRADNPGVWFMHCHLEVHTTWGLKMAFLVDNGKGPKESLLPPPSDLPKC

SEQ ID NO: 73 - protein, 557 aa, Populus sp.
MEYSWFRFMLLAVCLFPAVVECRIRHYKFNVVMKNTTRLCSSKPIVTVNGLFPGPTLYAREDDTVLVKVVNRVKYNLSIHWHGIRQLRTGWADGPAYITQCPIQPGQSYVYNFTITGQRGTLLWHAHILWLRATVHGALVVLPKLGVPYPFPAPHKEVVVVLAEWWKSDTEAVINEALKSGLAPNVSDAHTINGHPGAVSTCSSQGGFTLPVQSGKTYMLRLINAALNEELFFKIAGHKLTVVEVDATYVKPFKTDTVLIAPGQTTNVLVTTNKNTGKYLVAASPFMDAPIAVDNMTATATLHYSGALSNSPTTLTIPPPKNATAIANQFTNSLRSLNSKTFPAKVPLTVDHSLFFTVGLGINPCPTCKAGNGSRVVASINNVTFVMPTTALLQAHFFNISGVFTTDFPAKPPHVFNYTGTPPTNLQTTSGTKAYRLPYNSTVQLVMQDTGIISPENHPIHLHGFNFFAVGRGVGNYNPKTDPKKFNLVDPVERNTIGVPSGGWVAIRFRADNPGVWFMHCHLEVHTTWGLKMAFLVDNGKGPNESLLPPPSDLPKC

SEQ ID NO: 74 - protein, 564 aa, Populus sp.
MIRKSAKMEYSWFRFMLLAVSLFPALVECRVRHYKFNVVMKNTTRLCSSKPVVTVNGRFPGPTLYAREDDTVLVKVVNHVKYNVSIHWHGIRQLRTGWADGPAYITQCPIQTGQSYVYNFTITGQRGTLLWHAHILWLRATVHGAIVVLPKRGVPYPFPAPHKEFVVVLAEWWKSDTEAVINEALKSGLAPNVSDAHTINGHPGAVSACPSQGGFTLPVESGKTYMLRLINAALNEELFFKIAGHKLTLVEVDATYVKPFKTDTVLIAPGQTTNVLVTTNKNTGKYLVAASPFMDAPIAVDNMTATATLHYSGALSGTPTTLTIPPPKNATAVANQFTNSLRSLNSKRFPAKVPLTVDHNLFFTVGLGINPCPTCKAGNGSRVVASINNVTFVMPTTALLQAHFFNISGVFTTDFPSKPPHVFNYTGTPPTNLQTTSGTKVYRLRYNSTVELVMQDTGIISPENHPIHLHGFNFFGVGRGVGNYNPKTDPKKFNLVDPVERNTIGVPSGGWVAIRFRVDNPGVWFMHCHLEVHTTWGLKMAFLVDNGKGPNESLLPPPSDLPKC

SEQ ID NO: 75 - DNA, 107 bp, Arabidopsis thaliana
tgaatgaacatcattgagtgcagcgttgatgtaatttcgttttgtttttcattgttgaatggattaaaagaatttataccagcgttgcgctcaattatgtttttcta

SEQ ID NO: 76 - DNA, 109 bp, Arabidopsis thaliana
tgaatgaacatcattgagtgcatcgttgatgtaattttacttattttattccattgttgaattaattaaagaagtatatatcagcgttgcattcaattatgtttttcta

SEQ ID NO: 77 - DNA, 120 bp, Populus sp.
tggagaaccatcattgagtgcagcgttgatgaaatcctccattttgtgctattaaactgttaccaaccctttatggggcatggcatcatttcaccagcgctgcattcaatcatgtttttc

SEQ ID NO: 78 - DNA, 124 bp, Brachypodium distachyon
gaagaggcgcaaaggcatcattgagtgcagcgttgatgaacaggggccaggcgaccggcggccggtccggttcggttcaccggcgctgcacacagtgacgcccttgcattctctggcccgattc

SEQ ID NO: 79 - DNA, 45 bp, Brachypodium distachyon
agagggcaaggctcattggtgcagcgttgtgaacgggccgaccgg

SEQ ID NO: 80 - DNA, 13118 bp, Artificial
  Integrative vector
tttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagttttggagctccaactttgtatagaaaagttgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccagggggatcaccatccgtcgcccgggcgtgtcaataatatcactctgtacatccacaaacagacgataacggctctctcttttataggtgtaaaccttaaactgcatttcaccagcccctgttctcgtcggcaaaagagccgttcatttcaataaaccgggcgacctcagccatcccttcctgattttccgctttccagcgttcggcacgcagacgacgggcttcattctgcatggttgtgcttaccgaaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatcctctagattacgccccgcctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcaactttattatacatagttgataattcactggccgtcgttttacaagcttgtcggtacgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatcgatgagctcaaaacgtatgccgggccgctctagaactagagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtccctcccccctccccctccgccgccgccgcgccggtaacggccgctctagaactagtggatcccccgggccaggtccgtcgcttctcttccatttcttctcattttcgattttgattcttatttctttccagtagctcctgctctgtgaatttctccgctcacgatagatctgcttatactccttacattcaaccttagatctggtctcgattctctgtttctctgtttttttcttttggtcgagaatctgatgtttgtttatgttctgtcaccattaataataatgaactctctcattcatacaatgattagtttctctcgtctacaaaacgatatgttgcattttcacttttcttctttttttctaagatgatttgctttgaccaatttgtttagatctttattctattttattttctggtgggttggtggaaattgaaaaaaaaaaaaacagcataaattgttatttgttaatgtattcattttttggctatttgttctgggtaaaaatctgcttctactattgaatctttcctggattttttactcctattgggtttttatagtaaaaatacataataaaaggaaaacaaaagttttatagattctcttaaaccccttacgataaaagttggaatcaaaataattcaggatcagatgctctttgattgattcagatgcgattacagttgcatggcaaattttctagatccgtcgtcacattttattttctgtttaaatatctaaatctgatatatgatgtcgatcgacaaattctggtggcttatacatcacttcaactgttttcttttggctttgtttgtcaacttggttttcaatacgatttgtgatttcgatcgctgaatttttaatacaagcaaactgatgttaaccacaagcaagagatgtgacctgccttattaacatcgtattacttactactagtcgtattctcaacgcaatcgtttttgtatttctcacattatgccgcttctctactctttattccttttggtccacgcattttctatttgtggcaatccctttcacaacctgatttcccactttggatcatttgtctgaagactctcttgaatcgttaccacttgtttcttgtgcatgctctgttttttagaattaatgataaaactattccatagtcttgagttttcagcttgttgattcttttgcttttggttttctgcagaaacccgggctgcaggaattcctgcagatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcagccgctaccccgaccacatgaagcagcacgacttcttcaagtccgccatgcccgaaggctacgtccaggagcgcaccatcagcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacgtcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagagctcgaatttccccgatcgttcaaacatttgggaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcagtagcggccgcgtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctccttccatcccctcaaccctaccaccaccaccaccaccacctccacctcctcccccctggctgccggacgacgagctcctcccccctccccctccgccgccgccgcgccggtaaccaccccgcccctctcctctttctttctccgtttttttttttccgtctcggtctcgatctttggccttggtagtttgggtgggcgagaggcggcttcgtgcgcgcccagatcggtgcgcgggaggggcgggatctcgcggctggggctctcgccggtgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagacgataagcttgatatcgaattcctgcagcccggggatctaccatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgatgacccgggggatccactagttctagaggatccccgggtaccgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactgggataaattatcgcgcgcggtgtcatctatgttactagatcgggcggccgcctagacaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgaccgtaccccgatgaattaattcggacgtacgttctgaacacagctggatacttacttgggcgattgtcatacatgacatcaacaatgtacccgtttgtgtaaccgtctcttggaggttcgtatgacactagtggttcccctcagcttgcgactagatgttgaggcctaacattttattagagagcaggctagttgcttagatacatgatcttcaggccgttatctgtcagggcaagcgaaaattggccatttatgacgaccaatgccccgcagaagctcccatctttgccgccatagacgccgcgccccccttttggggtgtagaacatccttttgccagatgtggaaaagaagttcgttgtcccattgttggcaatgacgtagtagccggcgaaagtgcgagacccatttgcgctatatataagcctacgatttccgttgcgactattgtcgtaattggatgaactattatcgtagttgctctcagagttgtcgtaatttgatggactattgtcgtaattgcttatggagttgtcgtagttgcttggagaaatgtcgtagttggatggggagtagtcatagggaagacgagcttcatccactaaaacaattggcaggtcagcaagtgcctgccccgatgccatcgcaagtacgaggcttagaaccaccttcaacagatcgcgcatagtcttccccagctctctaacgcttgagttaagccgcgccgcgaagcggcgtcggcttgaacgaattgttagacattatttgccgactaccttggtgatctcgcctttcacgtagtgaacaaattcttccaactgatctgcgcgcgaggccaagcgatcttcttgtccaagataagcctgcctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactcctgaattaagccgcgccgcgaagcggtgtcggcttgaatgaattgttaggcgtcatcctgtgctcccgagaaccagtaccagtacatcgctgtttcgttcgagacttgaggtctagttttatacgtgaacaggtcaatgccgccgagagtaaagccacattttgcgtacaaattgcaggcaggtacattgttcgtttgtgtctctaatcgtatgccaaggagctgtctgcttagtgcccactttttcgcaaattcgatgagactgtgcgcgactcctttgcctcggtgcgtgtgcgacacaacaatgtgttcgatagaggctagatcgttccatgttgagttgagttcaatcttcccgacaagctcttggtcgatgaatgcgccatagcaagcagagtcttcatcagagtcatcatccgagatgtaatccttccggtaggggctcacacttctggtagatagttcaaagccttggtcggataggtgcacatcgaacacttcacgaacaatgaaatggttctcagcatccaatgtttccgccacctgctcagggatcaccgaaatcttcatatgacgcctaacgcctggcacagcggatcgcaaacctggcgcggcttttggcacaaaaggcgtgacaggtttgcgaatccgttgctgccacttgttaacccttttgccagatttggtaactataatttatgttagaggcgaagtcttgggtaaaaactggcctaaaattgctggggatttcaggaaagtaaacatcaccttccggctcgatgtctattgtagatatatgtagtgtatctacttgatcgggggatctgctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcagggggggggggggggggggacttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcccccccccccccccccctgcaggcatcgtggtgtcacgctcgtcg

(19) United States
(12) Patent Application Publication
(10) Pub. No.: US 2012/0192312 A1
(43) Pub. Date: Jul. 26, 2012

(54) Production of Plants with Reduced Lignin Content

(75) Inventors: Lise Jouanin, Noisy Le Roi (FR); Serge Berthet, Rombas (FR); Catherine Lapierre, Neauphle Le Chateau (FR); Emmanuel Guiderdoni, Aniane (FR); Oumaya Bouchabke-Coussa, Paris (FR); Richard Sibout, Jouy (FR); Jean-Charles Leple, Orleans (FR); Julien Mazel, Courdimanche (FR); Brice Ayangma, Tours (FR)

(73) Assignees: INSTITUT NATIONAL DE LA RECHERCHE AGRONOMIQUE, Paris (FR); GENOPLANTE-VALOR, Paris (FR)

(21) Appl. No.: 13/377,372

(22) PCT Filed: Jun. 10, 2010
(86) PCT No.: PCT/IB10/52590

§ 371 Date: Apr. 4, 2012

(30) Foreign Application Priority Data
Jun. 10, 2009 (FR) 0902812

(51) Int. Cl. C12N 15/82; D21H 11/12; A01H 5/00; C10L 1/00; C12N 5/10

(52) U.S. Cl. 800/284; 44/307; 435/419; 435/320.1; 800/298; 162/100

(57) ABSTRACT

The invention relates to the production of plants having a reduced lignin content and in which the cellulose hydrolysis of the walls is increased, via the total or partial inhibition of the expression and/or the activity of two laccases in said plant.

The present invention relates to a method for selecting or producing plants having a reduced lignin content.

Lignins are insoluble polymers which are located in plant walls and are the result of the polymerization of 3 phenolic monomers (or monolignols), deriving from the phenylpropanoid pathway (Neish, Constitution and Biosynthesis of Lignin, publisher New York: Springer Verlag, 1-43, 1968). Their biosynthetic pathway is complex and comprises various steps, one part of which is carried out in the cytoplasm (monolignol synthesis) and another part in the wall (polymerization). p-Coumaryl, coniferyl and sinapyl alcohols are the respective precursors for the p-hydroxyphenyl (H), guaiacyl (G) and syringyl (S) units constituting lignins. These precursors are oxidized to phenolic radicals which spontaneously couple via various linkages, thereby resulting in the formation of lignins. Among the inter-unit linkages, a distinction is made between labile linkages, called β-O-4 linkages, and resistant linkages. During polymerization, other linkages can also be established with other wall compounds (polysaccharides and proteins) in order to form a complex three-dimensional network. The formation of the phenolic radicals is thought to be catalyzed by oxidases, such as peroxidases, laccases or other oxidases. A large number of these enzymes in combination with regulatory proteins is thought to be necessary for assembly of the H, G and S units (Boudet, Plant Physiol. Biochem., 38, 81-96, 2000; see for review: Ralph et al., Lignins, Encyclopedia of Life Sciences, John Wiley & Sons, 2007). However, these enzymes are still poorly identified since they belong to multigene families (Barrière et al., Genes, Genomes and Genomics; Global Science Books, 2007, Review).

Although the mechanisms involved in vivo in lignin biosynthesis are not completely elucidated, it is generally considered that laccases could be volved in the first steps of polymerization, for the formation of dimers or trimers, while peroxidases could make it possible to obtain a greater degree of polymerization from the dimers and trimers (Ros Barcelo, International Review of Cytology, 176, 87-132, 1997).

The lignin content of plants has a major influence on their industrial uses. For example, it affects the nutritive value of plants intended for animal feed, and also the performance levels of papermaking processes (yield and quality of the paper pulp obtained) and the production yield for biofuel. Indeed:
an important component of the nutritional value of fodder plants, such as fodder corn, is the digestibility. Thus, cows fed with varieties that are more digestible show an increase in milk production and better weight gain. In addition, these more digestible varieties enable the animals to reach their potential with a lower level of supplementation, which makes it possible in particular to reduce production costs. An important factor limiting the digestibility of fodder plants is linked to the presence of lignins in the plant cell walls: the lignins establish various types of linkages with the other wall constituents (including polysaccharides) and hinder the accessibility of the digestive enzymes to the polysaccharides (carbohydrates), the main sources of energy for herbivores;
most paper pulp is obtained by chemical delignification of wood, in order to isolate the plant fibers consisting mainly of cellulose and linked together by the lignins. However, this delignification step is very demanding in terms of energy and reagents (acids in particular), owing to the resistance of lignins to chemical degradation. In the case of the thermomechanical production of paper pulp, the lignins which are not removed are responsible for light-induced yellowing of the paper. Lignins rich in S units are more sensitive to papermaking delignification processes since the S units are especially linked via β-O-4 linkages (linkages which are targets of these processes). Consequently, in the case of the hardwoods used by the papermaking industries, a higher content of β-O-4-linked S units is sought in the papermaking sector in order to improve the pulp-cooking yield (Guerra et al., Ind. Eng. Chem. Res., 47, 8542-8549, 2008);
biofuels are formed from bioethanol (a gasoline-miscible product) or from oil (for producing a diesel product). The production of bioethanol, currently carried out from starch or from sucrose, could also be carried out from wood cellulose or straw cellulose. In this case, the production process comprises the following steps: acid pretreatment of the raw material in order to break the interactions between lignins and polysaccharides (this pretreatment facilitates the action of hydrolytic enzymes which convert the wall polysaccharides into simple sugars), followed by a step of fermentation of the simple sugars, resulting in the production of bioethanol. However, the initial acid pretreatment leads to the production of compounds capable of inhibiting the fermentation step. It has therefore been suggested that, if the lignins were modified, the wall polysaccharides would be more accessible to hydrolytic enzymes. This would make it possible not only to eliminate or limit the acid pretreatment and the associated problems of fermentation inhibition, but also to reduce environmental impacts linked to the residues from the acid treatments.

In this context, the quantitative modification of the lignins in plants is the subject of numerous research studies. The qualitative modification of lignins (modification of their structure or of their interactions with the other wall polymers) is also greatly studied. For example, lignins rich in S units or in β-O-4 linkages are much easier to remove during the chemical production of paper pulp.

One of the preferred routes for decreasing the lignin content in plants concerns production by genetic engineering of plants. It has thus been proposed to act on the enzymes of the lignin biosynthesis pathway, such as laccases (International Application WO 97/45549), peroxidases (International Application WO 2004/080202), cinnamoyl CoA reductase (CCR; International Applications WO 97/12982 and WO 98/39454), caffeic acid O-methyl transferase (COMT; International Application WO 94/23044; Oba and Allen, J. Dairy Sci., 82, 135-142, 1999), cafeoyl coenzyme A 3-O-methyl transferase (CCoAOMT; Application EP 0516958; Guo et al., Transgenic Res. 10, 457-464, 2001), cinnamyl alcohol dehydrogenase (CAD; Lapierre et al., Plant

Physiol., 119, 153-164, 1999), and 4-coumarate:coenzyme A ligase (4CL; Hu et al., Nat. Biotech. 17, 808-812, 1999).

With regard more particularly to the laccases, International Application WO 97/45549 describes a tobacco laccase (the sequence of which is, moreover, described by Kiefer-Mayer et al., Gene, 178, 205-207, 1996), and proposes increasing or reducing the amount of lignins produced by a plant by overexpressing said laccase (or a protein having at least 50% of amino acids homologous to those of said laccase), or by inhibiting its expression.

In Arabidopsis thaliana, the laccase multigene family comprises 17 members, 7 of which are expressed in the stems, the stem being the most lignified organ. The genes most strongly expressed are LAC4 (At2g38080), LAC17 (At5g60020) and LAC2 (At2g29130). The LAC2 and LAC17 genes belong to the same subclass, and the LAC4 gene belongs to a subclass which is close according to the phylogenetic trees published by Pourcel et al. (Plant Cell, 17, 2966-2980, 2005) and Caparros-Ruiz et al. (Plant Science, 171, 217-225, 2006). The LAC17 gene encodes the LAC17 protein (AtLAC17), the sequence of which is available under accession number NM_{—}125395 in the Genbank database, and is also reproduced in the appended sequence listing under the identifier SEQ ID No. 2. The LAC4 gene encodes the LAC4 protein (AtLAC4), the sequence of which is available under accession number NM_{—}129364 in the Genbank database, and is also reproduced in the appended sequence listing under the identifier SEQ ID No. 4. The LAC2 gene encodes the LAC2 protein (AtLAC2), the sequence of which is available under accession number NM_{—}128470 (GI:186503951) in the Genbank database. In Arabidopsis, AtLAC17 is expressed in the interfascicular fibers.

The inventors have thus demonstrated that the proteins that are orthologs of the AtLAC17 protein exhibit at least 60% identity or at least 75% similarity with said protein and comprise, from the N-terminal end to the C-terminal end, at least one of the 4 consensus peptide domains of sequence:
H-W-H-G-I/V-R-Q-L (SEQ ID No. 12; amino acids corresponding to positions 80-87 of the peptide sequence of AtLAC17) or H-W-H-G-I/V-R/L-Q-L/M/V (SEQ ID No. 38),
I/V-N-A-A-L-N-D-E-L-F-F (SEQ ID No. 13; amino acids corresponding to positions 223-233 of the peptide sequence of AtLAC17) or I-N-A/S-A-L-N/E-D/N/E-E-L-F-F (SEQ ID No. 39),
E-S-H-P-L-H-L-H-G-F/Y-N/D-F-F-V-V-G-Q-G-F/Y-G-N-F/Y-D (SEQ ID No. 14; amino acids corresponding to positions 476-498 of the peptide sequence of AtLAC17), or E-S-H-P-L/F-H-L/M-H-G-F/Y-N/D-F/Y-F/Y-V-V/I-G-Q/T/E-G-F/V/T-G-N-F/Y-D/N (SEQ ID No. 40), and
A-D-N-P-G-V-W (SEQ ID No. 15; amino acids corresponding to positions 539-546 of the peptide sequence of AtLAC17), or A/V-D-N-P-G-V/ø-W/ø (SEQ ID No. 41), where “ø” indicates that an amino acid is absent, respectively.

By way of nonlimiting examples of orthologs of the A. thaliana LAC17 protein, mention will in particular be made of the laccases of:
corn (Zea mays) of sequences SEQ ID Nos. 5 and 6 (sequences also available under accession numbers NM_001112405.1 and EU957078 in the Genbank database) and the sequences available in the Genbank database under accession numbers GI:162461426 (SEQ ID No. 42), GI:226503958 (SEQ ID No. 43), GI:226494660 (SEQ ID No. 44), GI:212721074 (SEQ ID No. 45), GI:162463584 (SEQ ID No. 46) and GI:162461268 (SEQ ID No. 47),
sugar cane (Saccharum officinarum) of sequence SEQ ID No. 7,
sorghum (Sorghum bicolor) of sequences SEQ ID Nos. 8, 9, 10 and 11,
Brachypodium, such as the sequences Bradi1g66720 (SEQ ID No. 48), Bradi2g54680 (SEQ ID No. 49), Bradi1g24910 (SEQ ID No. 50), Bradi1g24880 (SEQ ID No. 51), Bradi2g54740 (SEQ ID No. 52), Bradi2g23370 (SEQ ID No. 53), Bradi2g23350 (SEQ ID No. 54) and Bradi2g54690 (SEQ ID No. 55),
rice, such as the sequences available in the Genbank database under accession numbers GI:113548170 (SEQ ID No. 56), GI:113534304 (SEQ ID No. 57), GI:113579298 (SEQ ID No. 58), GI:113579297 (SEQ ID No. 59), GI:113534303 (SEQ ID No. 60), GI:113579295 (SEQ ID No. 61), GI:255673866 (SEQ ID No. 62) (these sequences have the domain referenced under accession number IPR017761 in the InterPro database), and
poplar, such as the sequences PtLAC1 (gene POPTR_{—}0001s14010.1; SEQ ID No. 63), PtLAC40 (POPTR_{—}0001s41160.1; SEQ ID No. 64), PtLAC41 (POPTR_{—}0001s41170.1; SEQ ID No. 65), PtLAC6 (POPTR_{—}0001s41170.1; SEQ ID No. 66), PtLAC24 (POPTR_{—}0011s12090.1; SEQ ID No. 67) and PtLAC25 (POPTR_{—}0011s12100.1; SEQ ID No. 68).

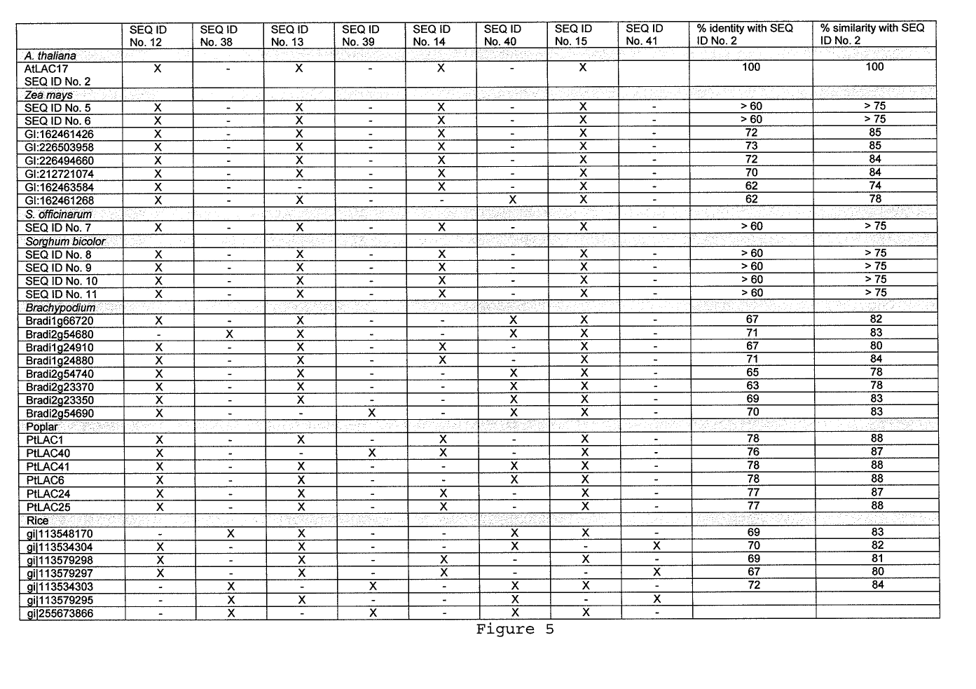

The table represented in FIG. 5 shows the presence (noted in the table by the sign “X”) of the consensus peptide domains as defined above in the sequences of the orthologs of the AtLAC17 protein in Zea mays, S. officinarum, Sorghum bicolor, Brachypodium, poplar and rice, and also the respective percentages of identity and of similarity relative to AtLAC17.

The LAC17 protein exhibits 55.2% identity with the LAC4 protein, and 67.1% identity with the LAC2 protein, but the latter does not comprise the consensus peptide domain of sequence SEQ ID No. 14, and 54.6% identity with the tobacco laccase described in International Application WO 97/45549; the LAC4 protein exhibits 54.0% identity with the LAC2 protein and 75.8% identity with the tobacco laccase described in International Application WO 97/45549 (it appears that said tobacco laccase is the ortholog of the AtLAC4 protein), the percentages of identity being calculated over the entire length of the sequences by means of the needle program (Needleman and Wunsch, J. Mol. Biol., 48, 443-453, 1970) using the default parameters: “Matrix”: EBLOSUM62, “Gap penalty”: 10.0 and “Extend penalty”: 0.5. In Arabidopsis, AtLAC4 is expressed in the vessels of the xylem and in the interfascicular fibers.

By way of nonlimiting examples of orthologs of the A. thaliana LAC4 protein, mention will in particular be made of the laccases of:
Brachypodium, such as the sequence Bradilg74320 (SEQ ID No. 69), which exhibits 67% identity and 83% similarity with the sequence SEQ ID No. 4,
rice, such as the sequence available in the Genbank database under accession number GI:150383842 (QOIQU1; SEQ ID No. 70), which exhibits 69% identity and 83% similarity with the sequence SEQ ID No. 4, and
poplar, the sequences PtLAC14 (POPTR_{—}0006s09830.1; SEQ ID No. 71, which exhibits 75% identity and 87% similarity with the sequence SEQ ID No. 4), PtLAC15 (POPTR_0006s09840.1; SEQ ID No. 72, which exhibits 76% identity and 87% similarity with the sequence SEQ ID No. 4), PtLAC32 (POPTR_{—}0016s11950.1; SEQ ID No. 73, which exhibits 78% identity and 88% similarity with the sequence SEQ ID No. 4) and PtLAC33 (POPTR_{—}0016s11960.1; SEQ ID No. 74, which exhibits 77% identity and 87% similarity with the sequence SEQ ID No. 4).

A. thaliana lines of the SALK collection in the Col0 accession (Columbia) exhibiting T-DNA insertions in the LAC17 (SALK_{—}016748 line), LAC4 (SALK_{—}051892 line) and LAC2 (SALK_{—}025690 line) genes have been identified. The mutants lac4 and lac2 have in particular been described by Brown et al. (Plant Cell, 17, 2281-2295, 2005). These mutants exhibit a greatly reduced or zero expression of the mutated gene, but do not exhibit any particular phenotype under glass.

The inventors have investigated whether these mutations have an effect on the amount of lignins of the mutated plants, and their qualitative (structural) properties. They have noted that the lac2 mutant does not exhibit any notable difference compared with the Col0 wild-type line, but that, on the other hand, the lac4 and lac17 mutants contain an amount of lignins (determined on mature dry stems) that is reduced by 6 to 8% and exhibit a cellulolysis yield that is increased by 17% in the case of lac17 and by 52% in the case of lac4, compared with the Col0 wild-type line (cellulolysis carried out without acid pretreatment).

In addition, the inventors have obtained lac4/lac17 double mutants from the lac4 and lac17 mutants by crossing. They have noted that these double mutants exhibit a very reduced amount of lignins (reduced by approximately 19% compared with the Col0 wild-type line), and a better cellulolysis yield compared with the Col0 wild-type line (+25% to +42%) and with the lac17 single mutant (+6% to +21%) approximately.

Consequently, the subject of the present invention is a method for reducing the lignin content of a plant and increasing the cellulolysis of the walls of said plant, characterized in that the expression and/or the activity in said plant:
a) of a laccase of which the polypeptide sequence has at least 60% identity, and in increasing order of preference at least 61%, 65%, 70%, 75%, 80%, 85%, 90%, 95%, 97%, 98% and 99% identity, or at least 75% similarity, and in increasing order of preference at least 78%, 79%, 80%, 83%, 85%, 90%, 95%, 97%, 98% and 99% similarity with the sequence SEQ ID No. 2 (LAC17), and comprises, from its N-terminal end to its C-terminal end, at least one of the 4, preferably at least 2 of the 4, more preferably at least 3 of the 4 and more preferentially the 4 consensus peptide domains i) to iv), respectively, which follow:
i) the consensus peptide domain of sequence SEQ ID No. 12 or 38,
ii) the consensus peptide domain of sequence SEQ ID No. 13 or 39,
iii) the consensus peptide domain of sequence SEQ ID No. 14 or 40,
iv) the consensus peptide domain of sequence SEQ ID No. 15 or 41, and
b) of a laccase of which the polypeptide sequence comprises at least 65% identity, and in increasing order of preference at least 70%, 75%, 80%, 85%, 90%, 95%, 97%, 98% and 99% identity, with the sequence SEQ ID No. 4 (LAC4),
   is totally or partially inhibited.

The term “lignin content” is intended to mean the Klason lignin content. This content can be measured by assaying the acid-insoluble lignin (ASL) fraction present in the parietal residue (PR) of a plant, as described in Example 3 below.

The term “laccase” is intended to mean a copper-comprising enzyme (EC 1.10.3.2) which catalyzes the oxidation of a phenolic substrate using dioxygen as final electron acceptor.

Unless otherwise specified, the percentages of identity indicated herein are established, as indicated above, by means of the needle program using the default parameters.

The present invention applies to dicotyledonous or monocotyledonous plants. By way of nonlimiting examples, it can apply to corn, wheat, barley, rye, triticale, oats, rice, sorghum, sugar cane, poplar and pine.

By way of nonlimiting examples of laccases, as defined in paragraph a) above, mention may be made, in corn (Zea mays), of the peptide sequences SEQ ID Nos. 5, 6 and 42 to 47, in sugar cane (Saccharum officinarum), of the sequence SEQ ID No. 7, in sorghum (Sorghum bicolor), of the peptide sequences SEQ ID Nos. 8 to 11, in Brachypodium, of the peptide sequences SEQ ID Nos. 48 to 55, in rice, of the peptide sequences SEQ ID Nos. 56 to 62, and in poplar, of the peptide sequences SEQ ID Nos. 63 to 68.

By way of nonlimiting examples of laccases, of which the polypeptide sequence has at least 60% identity or at least 75% similarity with the sequence SEQ ID No. 2 and comprises, from its N-terminal end to its C-terminal end, the 4 consensus peptide domains of sequence SEQ ID Nos. 12, 13, 14 and 15 respectively, mention may be made, in corn, of the peptide sequences SEQ ID Nos. 5, 6, 42, 43, 44 and 45, in sugar cane, of the peptide sequence SEQ ID No. 7, in sorghum, of the peptide sequences SEQ ID Nos. 8 (partial sequence of the protein), 9, 10 and 11, in Brachypodium, of the peptide sequences SEQ ID Nos. 50 and 51, in rice, of the peptide sequence SEQ ID No. 58, and in poplar, of the peptide sequences SEQ ID Nos. 63, 67 and 68.

By way of nonlimiting examples of laccases, of which the polypeptide sequence has at least 65% identity with the sequence SEQ ID No. 4, mention may be made, in Brachypodium, of the peptide sequence SEQ ID No. 69, in rice, of the peptide sequence SEQ ID No. 70, and in poplar, of the peptide sequences SEQ ID Nos. 71 to 74.

The total or partial inhibition of the expression and/or of the activity of a laccase as defined above can be obtained in various ways, by methods known per se.

Particularly advantageously, this inhibition can be obtained by intervening upstream of the production of said laccase, by mutagenesis of the gene encoding this protein, or else by inhibition or modification of the transcription or of the translation of this laccase.

The mutagenesis of the gene encoding said laccase can take place at the level of the coding sequence or of the expression-regulating sequences, in particular of the promoter. It is, for example, possible to delete all or part of said gene and/or to insert an exogenous sequence. By way of example, for corn, mention will be made of insertional mutagenesis: a large number of individuals deriving from a plant that is active in terms of the transposition of a transposable element (AC or mutator element) are produced, and the plants in which an insertion has taken place in the gene of said laccase are selected, for example by PCR. This exogenous sequence can also be a T-DNA (fragment of the Agrobacterium tumefaciens Ti plasmid).

It is also possible to introduce one or more point mutations with physical agents (for example radiation) or chemical agents. These mutations result in the reading frame being shifted and/or in a stop codon being introduced into the sequence and/or in the level of transcription and/or of translation of the gene being modified and/or in the enzyme being made less active than the wild-type protein. The mutated alleles of the gene of said laccase can be identified, for example, by PCR using primers specific for said gene.

In this context, techniques of “TILLING” type (Targeting Induced Local Lesions IN Genomes; McCallum et al., Plant Physiol., 123, 439-442, 2000) can in particular be used.

It is also possible to carry out a site-directed mutagenesis targeting the gene encoding said laccase. The inhibition or the modification of the transcription and/or the translation can be obtained via the expression of sense, antisense or double-stranded RNAs derived from the gene of said laccase, or of the cDNA of this protein, or else through the use of interfering RNAs (for review on antisense inhibition techniques see, for example: Watson and Grierson, Transgenic Plants: Fundamentals and Applications (Hiatt, A, ed) New York: Marcel Dekker, 255-281, 1992; Chicas and Macino, EMBO reports, 21, 992-996, 2001; for review concerning more specifically the use of interfering RNAs, see Hannon, Nature, 418, 244-251, 2002).

The subject of the present invention is also a recombinant DNA construct comprising one or more polynucleotides capable of inhibiting the expression of the two laccases as defined above. By way of nonlimiting examples, said polynucleotides can encode antisense RNAs, interfering RNAs (noncoding double-stranded RNAs approximately 21 to 25 nucleotides in length), micro-RNAs (noncoding, single-stranded RNAs approximately 21 to 25 nucleotides in length) (Ossowski et al., The Plant Journal, 53, 674-690, 2008; Schwab et al., Methods Mol Biol., 592, 71-88, 2010; Wei et al., Funct Integr Genomics., 9, 499-511, 2009) or ribozymes targeting the gene encoding a laccase as defined above.

Preferably, said polynucleotides capable of inhibiting the expression of the LAC4 and LAC17 laccases as defined above are micro-RNAs, such as the micro-RNAs miR397 and miR408, preferably miR397. By way of nonlimiting examples of such micro-RNAs, use may be made of those having the following sequences:
SEQ ID No. 75 (AtmiR397a) or SEQ ID No. 76 (AtmiR397b), obtained from Arabidopsis thalina (Abdel-Ghany and Pilon, J Biol Chem., 283, 15932-15945, 2008);
SEQ ID No. 77 (ptc-miR397), obtained from poplar; or
SEQ ID No. 78 (Bdi-miR397a) or SEQ ID No. 79 (Bdi-miR397b), obtained from Brachypodium distachyon (Zhang et al., BMC Genomics., 23, 10:449, 2009; Unver and Budak, Planta, 230, 659-669, 2009).

According to one preferred embodiment of the invention, the recombinant DNA construct is chosen from:
a DNA construct comprising a fragment of at least 15 consecutive nucleotides, preferably at least 20 consecutive nucleotides of the cDNA of the gene encoding a laccase as defined above,
a DNA construct of from 200 to 1000 bp, comprising a fragment of the cDNA of the gene encoding a laccase as defined above, or a complementary polynucleotide which, when it is transcribed, forms an RNA hairpin (or ribozyme) targeting said gene;
a DNA construct capable, when it is transcribed, of forming a micro-RNA targeting the gene encoding a laccase as defined above.

According to one particular embodiment of the invention, said recombinant DNA construct comprises a fragment of at least 15 consecutive nucleotides, preferably at least 20, and entirely preferably at least 50 consecutive nucleotides of a polynucleotide of sequence SEQ ID No. 1 or SEQ ID No. 3, or of a polynucleotide complementary to a polynucleotide of sequence SEQ ID No. 1 or SEQ ID No. 3.

These constructs can in particular be:
expression cassettes comprising one or more recombinant DNA constructs as defined above, under the transcriptional control of a suitable promoter. These expression cassettes can also advantageously comprise other regulatory elements, in particular regulatory elements for transcription, such as terminators, enhancers, etc.;
recombinant vectors comprising one or more recombinant DNA constructs as defined above, or advantageously an expression cassette as defined above.

Recombinant DNA constructs in accordance with the invention can also comprise other elements, for example one or more selectable markers.

Those skilled in the art have at their disposal a very wide choice of elements that can be used for obtaining recombinant DNA constructs in accordance with the invention.

By way of nonlimiting examples of promoters that can be used in the context of the present invention, mention will be made of:
constitutive promoters, such as the cauliflower mosaic virus (CaMV) 35S promoter described by Kay et al. (Science, 236, 4805, 1987), or derivatives thereof, the cassava vein mosaic virus (CsVMV) promoter described in International Application WO 97/48819, the maize ubiquitin promoter or the rice “actin-intron-actin” promoter (McElroy et al., Mol. Gen. Genet., 231, 150-160, 1991; GenBank accession number S 44221);
inducible or tissue-specific promoters, in order to modify the lignin content or composition only at certain developmental stages of the plant, under certain environmental conditions, or in certain target tissues, such as, for example, the stems, the leaves, the seeds, the spathes, the cortex or the xylem (e.g., the cinnamyl alcohol dehydrogenase (CAD) promoter or the 4-coumarate-CoA ligase (4CL) promoter).

By way of nonlimiting examples of other regulatory elements for transcription that can be used in the context of the present invention, mention will be made of terminators, such as the NOS 3′ terminator of nopaline synthase (Depicker et al., J. Mol. Appl. Genet., 1, 561-573, 1982), or the CaMV 3′ terminator (Franck et al., Cell, 21, 285-294, 1980; GenBank accession number V00141).

By way of nonlimiting examples of selectable marker genes that can be used in the context of the present invention, mention will in particular be made of genes which confer resistance to an antibiotic (Herrera-Estrella et al., EMBO J., 2, 987-995, 1983) such as hygromycin, kanamycin, bleomycin or streptomycin, or to a herbicide (EP 0 242 246) such as glufosinate, glyphosate or bromoxynil, or the NPTII gene which confers resistance to kanamycin (Bevan et al., Nucleic Acid Research, 11, 369-385, 1984).

The plants can be transformed using numerous methods, known in themselves to those skilled in the art.

It is, for example, possible to transform plant cells, protoplasts or explants and to regenerate a whole plant from the transformed material. The transformation can thus be carried out, by way of nonlimiting examples:
by transfer of the vectors in accordance with the invention into protoplasts, in particular after incubation of the latter in a solution of polyethylene glycol (PEG) in the presence of divalent cations (Ca²⁺) according to the method described in the article by Krens et al. (Nature, 296, 72-74, 1982);
by electroporation, in particular according to the method described in the article by Fromm et al. (Nature, 319, 791-793, 1986);
by using a gene gun which makes it possible to discharge, at very high speed, metal particles coated with the DNA sequences of interest, thus delivering genes inside the cell nucleus, in particular according to the technique described in the article by Finer et al. (Plant Cell Report, 11, 323-328, 1992);
by cytoplasmic or nuclear microinjection.

Use may also be made of Agrobacterium tumefaciens, in particular according to the methods described in the articles by Bevan et al. (Nucleic Acid Research, 11, 369-385, 1984) and by An et al. (Plant Phydiol., 81, 86-91, 1986), or else Agrobacterium rhizogenes, in particular according to the method described in the article by Jouanin et al. (Plant Sci., 53, 53-63, 1987). For example, the plant cell transformation can be carried out by transfer of the T region of the Agrobacterium tumefaciens tumor-inducing extrachromosomal circular plasmid Ti, using a binary system (Watson et al., publisher De Boeck University, 273-292, 1994). Agrobacterium tumefaciens can also be used on whole plants, for example by depositing at the level of the wound of a monocotyledonous plant, the bacterium harboring the DNA to be transferred, in the presence of substances released at the level of the wound of a dicotyledonous plant.

The subject of the present invention is also a plant cell comprising an expression cassette as defined above or a recombinant vector as defined above.

The subject of the present invention is also the plants which can be obtained by means of a method in accordance with the invention, with the exception of the A. thaliana mutants SALK_016748 and SALK_051892. Said plants can carry mutations which inhibit the LAC4 and LAC17 laccases as defined above or express one or more polynucleotides capable of inhibiting the expression of said LAC4 and LAC17 laccases as defined above. Of course, the present invention encompasses the descendants, in particular the hybrids resulting from crossing involving at least one plant according to the invention, which are obtained by sowing or by vegetative multiplication of the plants directly obtained by means of the method of the invention.

The plant material, such as protoplasts, cells, calluses, leaves, stems, roots, flowers, fruits, cuttings and/or seeds, obtained from the plants in accordance with the invention (with the exception of the A. thaliana mutants SALK_{—}016748 and SALK_{—}051892), are also part of the subject of the present invention.

The subject of the present invention is also the use of the plants in accordance with the invention or of plant material obtained from said plants, for producing fodder plants, biofuels or paper pulp.

The present invention will be understood more clearly by means of the further description which follows, which refers to nonlimiting examples illustrating the reduction in lignin content and the increase in cellulolysis of the walls of a plant in which the expression of the LAC17 and/or LAC4 laccases is inhibited, and also the appended figures:

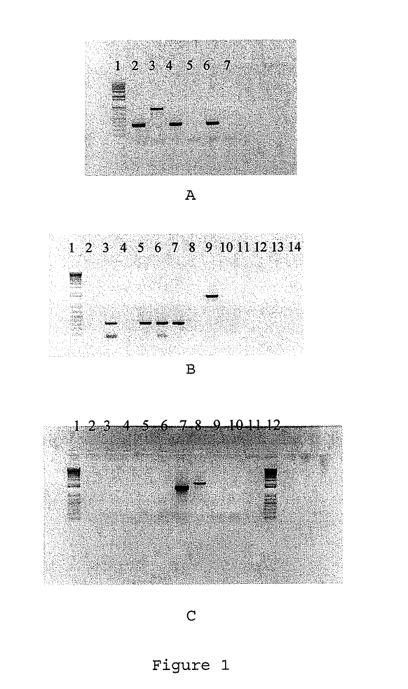

FIG. 1: analysis by 1% agarose gel electrophoresis of the PCR products obtained from genomic DNA of the A. thaliana lines SALK_{—}016748 (lac17 mutant) (Figure A), SALK_{—}051892 (lac4 mutant) (Figure B) and SALK_{—}025690 (lac2 mutant) (Figure C). A: well 1: 1 Kb+ size marker (Invitrogen); well 2: tubulin of the Col0 wild-type line; well 3: laccase 17 of the wild-type line; wells 4 and 6: tubulin of 2 plants of the SALK_{—}016748 line (lac17); wells 5 and 7: laccase 17 of 2 plants of the SALK_{—}016748 line (lac17). B: well 1: 1 Kb+ size marker (Invitrogen), well 2: empty; well 3: tubulin of the Col0 wild-type line; well 4: empty; wells 5 to 7: tubulin of 3 plants of the SALK_{—}051892 line (lac4); well 8: empty; well 9: laccase 4 of the Col0 wild-type line; well 10: empty; wells 11 to 13: laccase 4 of 3 plants of the SALK_{—}051892 line (lac4). C: wells 1 and 12: 1 Kb+ size marker (Invitrogen); wells 2 to 6 and 11: empty; well 7: laccase 2 of the Col0 wild-type line (cDNA); well 8: laccase 2 of 1 plant of the Col0 wild-type line (gDNA); well 9: laccase 2 of 1 plant of the SALK_{—}025690 line; well 10: control, laccase 2 amplified on the RNA of a plant of the Col0 wild-type line having undergone the same treatment as the other plants, with the difference that, during the reverse transcription step, the reverse transcriptase was not added;

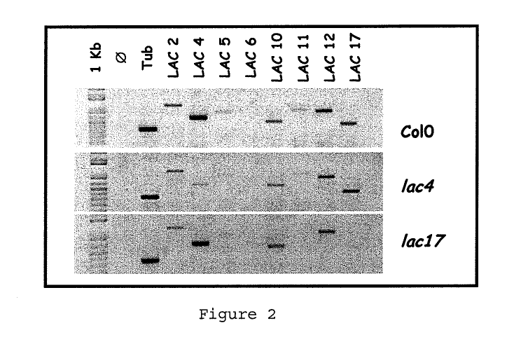

FIG. 2: analysis of the expression profile of A. thaliana laccases (LAC 2, 4, 5, 6, 10, 11, 12 and 17) by 1% agarose gel electrophoresis, in TAE buffer, of the RT-PCR products obtained from cDNA of the cells of the floral scape of the Col0 wild-type line, of the SALK_{—}051892 line (lac4) and of the SALK_{—}016748 line (lac17). Tub=tubulin;

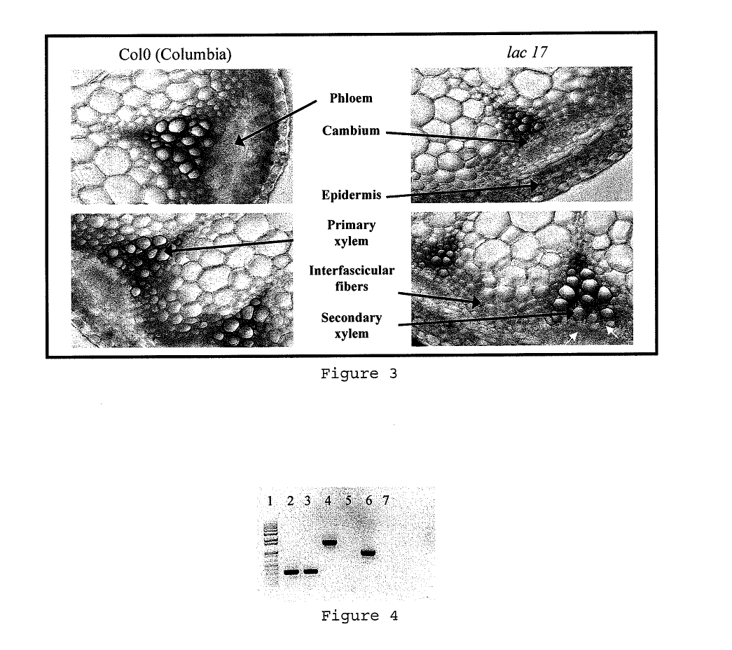

FIG. 3: optical microscope observation (200X magnification) of 70-micron-thick transverse sections primary scape of 20 cm, stained with phloroglucinol-HCl, from the Col0 wild-type line and from the SALK_{—}016748 line (lac17);

FIG. 4: analysis by 1% agarose gel electrophoresis of the PCR products obtained from genomic DNA of an A. thaliana double mutant Kim (lac4/lac17). Well 1: 1 Kb+ size marker (Invitrogen); well 2: tubulin of the wild-type line; well 3: tubulin of the Kim mutant; well 4: laccase 4 of the wild-type line; well 5: laccase 4 of the Kim mutant; well 6: laccase 17 of the wild-type line; well 7: laccase 17 of the Kim mutant.

### EXAMPLE 1

### Selection, Genotyping and Characterization of Arabidopsis thaliana lac17, lac4 AND lac2 Mutants

1) Selection of the A. thaliana Laccase Mutants

A. thaliana lines of the SALK collection in the Col0 accession exhibiting T-DNA insertions in the LAC17, LAC4 and LAC2 genes (respectively, the SALK_{—}016748, SALK_{—}051892 and SALK_{—}025690 lines) were identified and characterized.

The SALK_{—}016748 (lac17) mutant contains two T-DNAs inserted in inverted tandem into the promoter of the gene encoding LAC17, 146 base pairs from the ATG start codon.

The SALK_{—}051892 (lac4) mutant contains one T-DNA inserted into the promoter of the gene encoding LAC4, 127 base pairs from the ATG start codon.

The SALK_{—}025690 (lac2) mutant contains one T-DNA inserted into its coding sequence.

2) Genotyping of the A. thaliana lac17 (SALK_{—}016748), lac4 (SALK_{—}051892) and lac2 (SALK_{—}025690) Mutants

a) Materials and Methods

Primers for the LAC17, LAC4 and LAC2 genes were defined using the OLIGO 4 software (National Biosciences Inc., Plymouth, USA). Their sequences (5′->3′) are represented in Table 1 hereinafter:

TABLE 1

| | Sense primer | Antisense primer |
|---|---|---|
| | sequence | sequence |
| Primers for | Lac 17 FST dir: | Lac 17 FST rev: |
| LAC17 gene | TCG AAG AGG GTC AAA | TCT TAG CCA TGA |
| amplification | GAG TTT (SEQ ID | AAT GTG AGC (SEQ |
| | No. 16) | ID No. 17) |
| Primers for | irx12 FST dir: | irx12 FST rev: |
| LAC4 gene | ATT GTG TAA GCA AAT | TGG CTT GCT TGA |
| amplification | CGG CAC (SEQ ID | GCA TAA TCT (SEQ |
| | No. 18) | ID No. 19) |
| Primers for | Lac 2 RT dir: | Lac 2 RT rev: |
| LAC2 gene | GCA AGA CAA AAA CAA | GAA ATC TGA GGG |
| amplification | TCG TGA (SEQ ID | TGG AGG AAG (SEQ |
| | No. 20) | ID No. 21) |

The DNA of the plants was extracted according to the protocol described by Edwards et al. (Nucleic Acid Research, 19, 1349, 1991).

The PCRs were carried out in 25 μl on 30 ng of genomic DNA, with 2 mM of MgCl₂, 0.4 mM of each dNTP, 0.4 mM of each primer, and 1.25 units of Taq DNA polymerase (Invitrogen).

The PCR cycles for the genotyping of the lac17 mutants are (95° C. 30 sec, 50° C. 30 sec, 72° C. 1 min) 28 times, with a final extension of 10 min at 72° C.

The PCR cycles for the genotyping of the lac4 mutants are (95° C. 30 sec, 58° C. 30 sec, 72° C. 30 sec) 30 times, with a final extension of 10 min at 72° C.

The PCR cycles for the genotyping of the lac2 mutants are (95° C. 30 sec, 54° C. 30 sec, 72° C. 1 min 30 sec) 30 times, with a final extension of 10 min at 72° C.

The PCR products are then separated on 1% agarose gels in TAE buffer.

b) Results

2 plants of the SALK_{—}016748 line, 3 plants of the SALK_051892 line and 1 plant of the SALK_{—}025690 line were tested.

The genotyping results are represented in FIG. 1:
the 2 plants of the SALK_{—}016748 line that were tested are homozygous for the mutation in the LAC17 gene: the presence of the T-DNAs on the two strands coding for this gene prevents the amplification of a fragment of the LAC17 gene (FIG. 1A);
the 3 plants of the SALK_{—}051892 line that were tested are homozygous for the mutation in the LAC4 gene: the presence of the T-DNA on the two strands coding for this gene prevents the amplification of a fragment of the LAC4 gene (FIG. 1B);
the plant of the SALK_{—}025690 line that was tested is homozygous for the mutation in the LAC2 gene: the presence of the T-DNA on the two strands coding for this gene prevents the amplification of a fragment of the LAC2 gene (FIG. 1C).

3) Laccase Expression in the lac17 and lac4 Mutants

The expression profile of Arabidopsis laccases expressed in the floral scape in the wild-type line (Columbia), the lac17 mutant (SALK_{—}016748) and the lac4 mutant (SALK_{—}051892) was determined by RT-PCR.

a) Materials and Methods

A 26-cycle RT-PCR was performed on cDNAs obtained from 1 μg of RNA extracted using an RNeasy kit (Qiagen), that were treated with a DNase and reverse-transcribed with the SSRTII reverse transcriptase (Invitrogen).

The primers used are described in Table 2 hereinafter:

TABLE 2

| | | | Tm (° C.) |
|---|---|---|---|
| Gene | Name of | | of the |
| amplified | primer | Sequence (5′->3′) | primer |
| LAC2 | LAC 2RT | GCA AGA CAA AAA CAA TCG TGA | 58 |
| | dir | SEQ ID No. 20 | |
| LAC2 | LAC 2RT | GAA ATC TGA GGG TGG AGG AAG | 64 |
| | rev | SEQ ID No. 21 | |
| LAC4 | lac4 FST | AGT AAT GAA CAG TTGCGG TGG | 62 |
| | dir | SEQ ID No. 22 | |
| LAC4 | lac4 FST | TGG TAA CTT TGG ACG ATC AGG | 58 |
| | rev | SEQ ID No. 23 | |
| LAC4 | LAC 4 RT | GTT AGA AAC TGT CCA TCT CAA | 58 |
| | dir | SEQ ID No. 24 | |
| LAC4 | LAC 4 RT | CTC CAC TTG TGT TGA AGT AAT | 58 |
| | rev | SEQ ID No. 25 | |
| LAC4 | irx 12 FST | ATT GTG TAA GCA AAT CGG CAC | 60 |
| | dir | SEQ ID No. 18 | |
| LAC4 | irx 12 FST | TGG CTT GCT TGA GCA TAA TCT | 60 |
| | rev | SEQ ID No. 19 | |
| LRC5 | LAC 5 RT | ATC CGG TTG ATG TGT TGA GA | 58 |
| | dir | SEQ ID No. 26 | |
| LAC5 | LAC 5 RT | AGA GAG ATC GGC TTA TGT TG | 58 |
| | rev | SEQ ID No. 27 | |
| LAC6 | LAC 6 RT | TAT GCC AAA CAA ACG AGA T | 52 |
| | dir | SEQ ID No. 28 | |
| LAC6 | LAC 6 RT | CTG CTG GAG GAG GAG GTC | 60 |
| | rev | SEQ ID No. 29 | |
| LAC10 | LAC 10 RT | TGT AAA GCC GGA AAC TTC TC | 58 |
| | dir | SEQ ID No. 30 | |
| LAC10 | LAC 10 RT | TTA GGG CCT TTA CCA TTC TC | 58 |
| | rev | SEQ ID No. 31 | |
| LAC11 | LAC 11 RT | GAG CTA TTC TTC GGG ATT | 52 |
| | dir | SEQ ID No. 22 | |
| LAC11 | LAC 11 RT | GTC TTT AGG CGG TGG TAG | 56 |
| | rev | SEQ ID No. 33 | |
| LAC12 | LAC 12 RT | GCC GAC GCA TCT TAC CTC | 58 |
| | dir | SEQ ID No. 34 | |
| LAC12 | LAC 12 RT | CCA AGA ACG CCA TAG CAA | 54 |
| | rev | SEQ ID No. 35 | |
| LAC17 | lac17 FST | TCG AAG AGG GTC AAA GAG TTT | 60 |
| | dir | SEQ ID No. 16 | |
| LAC17 | lac17 FST | TCT TAG CCA TGA AAT GTG AGC | 60 |
| | rev | SEQ ID No. 17 | |
| LAC17 | LAC 17 RT | TTC TCT TGT GTT CTT CTT CTT | 56 |
| | dir | SEQ ID No. 36 | |
| LAC17 | LAC 17 RT | GAA CTT CTT TGT GAG GTT TAG | 58 |
| | rev | SEQ ID No. 37 | |

The pairs of primers termed “FST” (for Flanking Sequence Tag) were designed on either side of the T-DNA; they were used for amplifying on gDNA (genomic DNA). The pairs of primers termed “RT” were defined in the coding sequence and make it possible to amplify on cDNAs.

The RT-PCR cycles on the lac17 and lac4 mutants and the wild-type line are (95° C. 30 sec, 50° C. 30 sec, 72° C. 1 min 30) 26 times, with a final extension of 10 min at 72° C. for the laccases 2, 4, 6, 12 and 17 and the tubulins.

The RT-PCR cycles on the lac17 and lac4 mutants and the wild-type line are (95° C. 30 sec, 55° C. 30 sec, 72° C. 1 min 30) 26 times, with a final extension of 10 min at 72° C. for the laccases 5 and 10.

The RT-PCR cycles on the lac17 and lac4 mutants and the wild-type line are (95° C. 30 sec, 58° C. 30 sec, 72° C. 1 min 30) 26 times, with a final extension of 10 min at 72° C. for the laccase 11.

The RT-PCR products are then separated on 1% agarose gels in TAE buffer in order to visualize a difference in intensity of the fragments amplified.

b) Results

The results are represented in FIG. 2. Only the level of the laccase 17 (LAC17) transcripts decreases in the lac17 mutant and that of laccase 4 (LAC4) in the lac4 mutant. The mutants do not overexpress any other laccase for the purpose of compensating for the loss of expression of LAC17 and LAC4.

4) Cytological Analysis of the lac17 Mutant

a) Materials and Methods

Sections of primary scape of 20 cm were stained with phloroglucinol-HCl according to the protocol described by Sibout et al. (Plant Cell, 17, 2059-2076, 2005). The red coloration observed corresponds to the lignified cell walls.

b) Results

The cytological observation results are represented in FIG. 3. A delay and/or a decrease in the amount of lignin deposited on the cell walls is observed in the lac17 mutant but not in the wild-type line (FIG. 3).

### EXAMPLE 2

### Production and Molecular Characterization of an Arabidopsis thaliana lac17/lac4 Double Mutant

a) Materials and Methods

Plants of the SALK_{—}016748 line (lac17) were crossed with plants of the SALK_{—}051892 line (lac4) in order to obtain a lac4/lac17 double mutant (hereinafter referred to as Kim mutant).

The lac4/lac17 double mutant was then characterized by genotyping according to the protocol described in Example 1.a) and using the lac4 FST dir, lac4 FST rev, irx12 FST dir and irx12 FST rev primers.

b) Results

The results of the genotyping for the Kim mutant are represented in FIG. 4: the Kim mutant is homozygous for the mutations in the LAC17 and LAC4 genes. Indeed, the presence of the T-DNAs on the two strands coding for these genes prevents the amplification of a fragment of the LAC17 and LAC4 genes.

The presence of two T-DNAs in the promoter of the LAC17 gene and of one T-DNA in the promoter of the LAC4 gene was confirmed by amplification of the sequences adjacent to the T-DNAs and sequencing of the amplicons.

### EXAMPLE 3

### Analysis of the Lignin Content and of the cellulolysis of the Arabidopsis thaliana lac17 and lac4 Single Mutant and lac17/lac4 Double Mutant

a) Materials and Methods

i) Assaying of Lignin Content

The assaying of the lignins was carried out on the stems collected at maturity, ground and subjected to thorough extraction with the solvents ethanol/toluene (2/1, v/v), ethanol, and then water; extractions carried out in a Soxhlet apparatus. The material extracted and dried represents the “parietal residue” or PR (since it consists of the plant walls). The removal of the soluble compounds by extraction with solvent is essential before any assaying of lignins (these compounds possibly interfering with gravimetric or spectrometric assays).

The lignin content was measured by assaying the acid-insoluble lignin fraction present in the PR and called Klason lignin (KL). This KL fraction, assayed by gravimetric analysis by treating the parietal residue with concentrated sulfuric acid (which makes it possible to hydrolyze the polysaccharides and to leave a KL residue which is rinsed, dried and weighed), represents most of the parietal lignins. However, a very small fraction of the lignins may be solubilized during the treatment with sulfuric acid: it is the fraction called acid-soluble lignin (ASL), which is evaluated by measuring the absorbance of the sulfuric supernatant in the ultraviolet range.

These measurements were carried out using the T222 om-83 method, known to those skilled in the art, and developed for wood and derivatives thereof by TAPPI (Technical Association of the Pulp and Paper Industry) (C. W. Dence, The determination of lignin; in: S. Y. Lin, and C. W. Dence, (Eds.). Methods in Lignin Chemistry. Springer-Verlag, pp. 33-61, 1992).

ii) Study of Lignin Structure

The structure of the lignins was evaluated by thioacidolysis. Thioacidolysis of the lignins releases thioethylated monomer products H, G or S from the p-hydroxyphenyl (H), guaiacyl (G) or syringyl (S) units linked only via β-O-4 linkages (major inter-unit linkages in native lignins). These products were analyzed by gas chromatography coupled to mass spectrometry (GC-MS) of their trimethylsilyl (TMS) derivatives. The trimethylsilyl H, G or S monomers were assayed using chromatograms reconstructed respectively on the 239, 269 or 299 ions (the most intense ions of their mass spectrum obtained by electron impact).

The protocol that was used is similar to that described by Lapierre et al. (Res. Chem. Interm., 21, 397-412, 1995) and by Mir Derikvand et al. (Planta 227, 943-956, 2008). The H monomers are most commonly minor (less than 1% of the total monomers) and were therefore not considered (except in the case of mutant plants affected in the formation of the G and S units, or in the case of stress lignins).

iii) Measurement of the Enzymatic Degradability by Cellulolysis in Vitro

The susceptibility of the parietal polysaccharides to enzymatic hydrolysis was evaluated in vitro, by subjecting the walls (i.e. the parietal residue PR) to a preparation of cellulase and hemicellulase enzymes.

The protocol that was used is described by Hoffmann et al. (Plant Cell 16, 1446-1465, 2004), which is a protocol adapted from the method by Rexen (Anim. Feed Sci. Technol., 2, 205-218, 1977). Said enzymatic preparation used was the Cellulase Onozuka R10 preparation extracted from Trichoderma viride, 096i/mg (Serva Electrophoresis GmbHD, Germany).

b) Results

The results are given in Table 3 (giving the mean values between the 2 analytical repeats and the mean deviation between these repeats, except for the percentage PR for which a single measurement was carried out) hereinafter, in which:
% PR=percentage of parietal residue. This percentage reflects the amount of wall in the dry sample (% by weight of the dry sample); it is given by way of indication;
% KL=amount of Klason lignin expressed as % by weight of the PR;
m.d. KL=mean deviation between 2 independent analytical KL measurement repeats;
% ASL=amount of acid-soluble lignin expressed as % by weight of the PR and measured on the basis of the absorbance at 205 nm of the sulfuric supernatant resulting from the measurement of the Klason lignin (using an absorptivity coefficient of 110 1.g⁻¹.cm⁻¹);
m.d. ASL=mean deviation between 2 independent analytical ASL measurement repeats;
yld thio μmol/g KL=yield of monomers (G+S) from thioacidolysis, expressed in μmol per gram of Klason lignin (the H monomers obtained in trace amounts are not considered). When it is calculated on the basis of the Klason lignin content, the total yield of monomers from thioacidolysis reflects the proportion of units linked only via β-O-4 linkages in the lignins. This structural information is important since it reflects the susceptibility of the lignins to industrial delignification processes;
m.d. yld thio=mean deviation of the yield from thioacidolysis between 2 independent analytical thioacidolysis repeats;
S/G thio=molar ratio of the G and S monomers released by thioacidolysis of the lignins. This ratio reflects the proportion of S units and of G units in the native lignins. In angiosperms, the extent of the S units varies according to the developmental stage (the S units being deposited mainly at the end of lignification) and also to the tissues (the fibers are richer in S units than the vessels). Consequently, when a mutant plant has an S/G ratio that is different than that of the control line (cultivated under the same conditions), this difference may be attributable to the fact that the mutation affects lignification over time (at the start or at the end) or in a tissue-specific manner (affects fiber lignification or vessel lignification). It is also possible that the mutation affects more specifically an enzyme involved in the biosynthesis of coniferyl alcohol (precursor of G units) or of sinapyl alcohol (precursor of S units);
m.d. S/G=mean deviation of the thioacidolysis S/G molar ratio between 2 independent analytical thioacidolysis repeats;
% loss by cellulolysis=loss by weight (as %) of the PR treated with a commercial preparation of cellulase and hemicellulase enzymes;
m.d. cellulolysis=mean deviation between 2 independent analytical cellulolysis repeats;
the Kim 1 and Kim 2 mutants are 2 biological repeats of the same line. Kim 1 and Kim 2 were cultivated on 2 separate culture trays, which may explain the variations between these repeats.

It should be noted that the SALK_{—}025690 mutant (lac2) does not exhibit any decrease in the amount of lignins compared with the wild-type line (Col0).

TABLE 3
Study of the lignins of the scapes of the wild-type line (Col0) and the lines
mutant for laccases 4 and/or 17.

| | | | | | | yld | | | | | |
|---|---|---|---|---|---|---|---|---|---|---|---|
| | | | | | | thio | m.d. | | | | |
| | % | % | m.d. | % | m.d. | μmol/g | yld | S/G | m.d. | % loss by | m.d. |
| Lines | PR | KL | KL | ASL | ASL | KL | thio | thio | S/G | cellulolysis | cellulolysis |
| Col0 (wild-type) | 62.0 | 16.91 | 0.16 | 2.43 | 0.04 | 1261 | 50 | 0.46 | 0.00 | 26.9 | 1.0 |
| SALK_ 051892 | 61.4 | 15.51 | 0.07 | 1.76 | 0.03 | 1221 | 20 | 0.47 | 0.00 | 40.9 | 1.1 |
| (lac4) | | | | | | | | | | | |
| SALK_ 016748 | 61.4 | 15.81 | 0.08 | 2.05 | 0.04 | 1291 | 63 | 0.56 | 0.01 | 31.5 | 1.4 |
| (lac17) | | | | | | | | | | | |
| Kim 1 | 60.3 | 13.62 | 0.12 | 2.38 | 0.01 | 1265 | 90 | 0.65 | 0.01 | 38.1 | 1.5 |
| (lac4/lac17) | | | | | | | | | | | |
| Kim 2 | 62.6 | 13.73 | 0.09 | 2.41 | 0.08 | 1370 | 6 | 0.63 | 0.00 | 33.5 | 1.0 |
| (lac4/lac17) | | | | | | | | | | | |

It emerges from the above results that:
the homogeneity of the PR contents (60.3 to 62.6%) indicates that the mutations do not affect the lignocellulose-wall content of the mature stems. Since the mutant lines do not exhibit any reduction in size, this suggests that the mutant plants have the same productivity in terms of lignocellulose biomass that can be exploited as fibers or as biofuel, for example;
on the other hand, all the mutants exhibit a significantly lower Klason lignin content than that of the control: the decrease is moderate for the single mutants (decrease of 8 and 6% for the lac4 and lac17 mutants, respectively) and more marked for the double mutants (approximately 19%). This decrease in lignin content, which does not affect the growth and the development of the plant, is sought-after in the context of the chemical production of paper pulp from angiosperm lignocelluloses. It also facilitates enzymatic hydrolysis, the lignins acting as barriers between the enzymes and the polysaccharides;
the acid-soluble lignin (ASL) content of the mutant lines is lower than the ASL content of the wild-type line. The reduced Klason lignin (or acid-insoluble lignin) content is not therefore compensated for by an increase in acid-soluble lignin;
the yields from thioacidolysis, calculated on the basis of the KL content, are close between the wild-type line and the mutant lines. This result indicates that the lignins of the mutant lines contain as many labile linkages as the lignins of the wild-type lines. The mutations have not therefore accentuated the frequency of the resistant inter-unit linkages, which would be disadvantageous in the perspective of the chemical production of paper pulp, for example;
on the other hand, the single and double mutants exhibiting the lac17 mutation have a higher S/G ratio compared with that of the wild-type line. This result suggests that the lac17 mutation could affect the start of lignification (and therefore the depositing of the G units) and/or the lignification of the vessels (richer in G units than the fibers);
the yield from cellulolysis of the mutant lines is increased compared with that of the wild-type line. The decrease in Klason lignin content therefore improves the efficiency of the cellulolysis.

### EXAMPLE 4

### Production of Transgenic poplars Overexpressing a Micro-RNA capable of Inhibiting the Expression of Laccases LAC17 and LAC4

Two genetic constructs are prepared in order to overexpress a micro-RNA (called miR397, SEQ ID No. 77) capable of inhibiting the expression of poplar (Populus) laccases LAC17 and LAC4, under the control either of the CaMV 2x35S constitutive promoter or of the “lignin-specific” promoter of Eucalyptus cinnamyl alcohol dehydrogenase 2 (CAD2) (called EuCAD).

The Gateway pMDC32 binary vector (Curtis and Grossniklaus, Plant Physiology, 133, 462-469, 2003) is used to overexpress the transgenes under the control of the 2x35S constitutive promoter.

For expression under the control of the EuCAD promoter, the 2x35S promoter is excised from the pMDC32 plasmid above by digestion with the HindIII-KpnI enzymes (unique restriction sites) and replaced with the “lignin-specific” EuCAD promoter.

The genetic transformation of the poplar is carried out according to the method described in Leplé et al. (Plant Cell Rep. 11, 137-141, 1992), i.e. by coculture of explants of poplar stems with agrobacteria containing a binary vector for the expression of miR397, isolation of transgenic calluses and regeneration of transformed seedlings.

Transgenic calluses are selected for the regeneration step. Seedlings are regenerated from these different calluses, therefore corresponding to different transformation events. Each seedling is cloned by multiplication.

An example of each transgenic line is used to:
study the structure of the lignins by thioacidolysis (see above) on a stem fragment;
identify spatial variations in amount of lignins, by FTIR-ATR (“Fourier Transform InfraRed Spectroscopy—Attenuated Total Reflectance”) infrared imaging and histochemical staining with phloroglucinol.

These first phenotyping analyses make it possible to identify the lines which exhibit reductions in the amount of lignins in the wood.

Lines are then analyzed for the expression of the transgenes and the genes encoding LAC17 and LAC4. These expression analyses are carried out by quantitative RT-PCR (qRT-PCR).

### EXAMPLE 5

### Production of Transgenic Brachypodium distachyon Overexpressing a Micro-RNA Capable of Inhibiting the Expression of Laccases LAC17 AND LAC4

Gateway binary vectors which are compatible and specific for the transformation of monocotyledons, containing a sequence encoding a micro-RNA of sequence SEQ ID No. 78 or 79, under the control either of a constitutive promoter, for example the maize ubiquitin promoter (ZmUbi), or a “lignin-specific” promoter, and a selectable gene such as the pat gene (which confers resistance to basta), are used for the genetic transformation of Brachypodium distachyon.

The analysis of the structure and of the lignin content of the transgenic plants obtained can be carried out using the same methods as for the analysis of the transgenic poplars above.

### EXAMPLE 6

### Production of Transgenic Corn Overexpressing a Micro-RNA Capable of Inhibiting the Expression of Laccases LAC17 AND LAC4

A vector as described for the genetic transformation of Brachypodium can be used for the genetic transformation of corn.

It is also possible to use the integrative vector L1038 (represented by the sequence SEQ ID No. 80), which contains an expression cassette comprising a herbicide resistance gene (Basta resistance gene), an expression cassette comprising a gene encoding a fluorescent protein for following the transgene without genotyping (gene encoding a GFP under the control of an Actin promoter), and a “triple Gateway” cassette attR4-ccdB-attR3 (where attR4 and attR3 are recombination sites and ccdB is a negative selection gene), which makes it possible to recombine a promoter of choice (attL4-attR1 ends), a gene of choice (attL1-attL2 ends) (in the case in point, a micro-RNA miR397) and a mock (attR1-attL3) (see the instruction manual published by Invitrogen, “MultiSite Gateway Pro”, Version B, Oct. 3, 2006).

1. A process for reducing the lignin content of a plant and increasing the cellulolysis of the walls of said plant, wherein the expression and/or the activity in said plant:
a) of a laccase of which the polypeptide sequence has at least 60% identity or at least 75% similarity with the sequence SEQ ID No. 2, and comprises, from its N-terminal end to its C-terminal end, at least one of the four consensus peptide domains i) to iv), respectively, which follow:
i) the consensus peptide domain of sequence SEQ ID No. 12 or 38,
ii) the consensus peptide domain of sequence SEQ ID No. 13 or 39,
iii) the consensus peptide domain of sequence SEQ ID No. 14 or 40,
iv) the consensus peptide domain of sequence SEQ ID No. 15 or 41, and
b) of a laccase of which the polypeptide sequence has at least 65% identity with the sequence SEQ ID No. 4,
is totally or partially inhibited.

2. The process as claimed in claim 1, wherein the total or partial inhibition of the expression and/or the activity in said plant of said laccases is obtained by mutagenesis of the gene encoding these laccases, or else by inhibition or modification of the transcription or of the translation of these laccases.

3. The process as claimed in claim 1, wherein said laccase as defined in paragraph a) is:
in corn (Zea mays), chosen from the peptide sequences SEQ ID Nos. 5, 6 and 42 to 47,
in sugar cane (Saccharum officinarum), the sequence SEQ ID No. 7,
in sorghum (Sorghum bicolor), chosen from the peptide sequences SEQ ID Nos. 8 to 11,
in Brachypodium, chosen from the peptide sequences SEQ ID Nos. 48 to 55,
in rice, chosen from the peptide sequences SEQ ID Nos. 56 to 62, and
in poplar, chosen from the peptide sequences SEQ ID Nos. 63 to 68.

4. The process as claimed in claim 1, wherein said laccase as defined in paragraph b) is:
in Brachypodium, the peptide sequence SEQ ID No. 69,
in rice, the peptide sequence SEQ ID No. 70, and
in poplar, chosen from the peptide sequences SEQ ID Nos. 71 to 74.

5. A recombinant DNA construct, comprising one or more polynucleotides capable of inhibiting the expression of a laccase of which the polypeptide sequence has at least 60% identity or at least 75% similarity with the sequence SEQ ID No. 2, and comprises, from its N-terminal end to its C-terminal end, at least one of the 4 consensus peptide domains i) to iv), respectively, which follow:
i) the consensus peptide domain of sequence SEQ ID No. 12 or 38,
ii) the consensus peptide domain of sequence SEQ ID No. 13 or 39,
iii) the consensus peptide domain of sequence SEQ ID No. 14 or 40,
iv) the consensus peptide domain of sequence SEQ ID No. 15 or 41, and
of a laccase of which the polypeptide sequence has at least 65% identity with the sequence SEQ ID No. 4.

6. The DNA construct as claimed in claim 5, wherein said polynucleotide encodes an antisense RNA, an interfering RNA, a micro-RNA or a ribozyme targeting the genes encoding said laccases.

7. An expression cassette, comprising one or more DNA constructs as defined in claim 5, under the transcriptional control of a suitable promoter.

8. A recombinant vector, comprising one or more DNA constructs as defined in claim 5.

9. A plant cell, comprising an expression cassette as defined in claim 7.

10. A plant which can be obtained by means of the method as claimed in claim 1, with the exception of the Arabidopsis thaliana lines SALK_{—}016748 and SALK_{—}051892.

11. The use of a plant which can be obtained by means of the method as claimed in claim 1, or of plant material obtained from said plant, for producing fodder plants, biofuel or paper pulp.

12. The process as claimed in claim 1 wherein said laccase as defined in paragraph a) is:
in corn (Zea mays), chosen from the peptide sequences SEQ ID Nos. 5, 6, 42, 43, 44 and 45,
in sugar cane (Saccharum officinarum), the sequence SEQ ID No. 7,
in sorghum (Sorghum bicolor), chosen from the peptide sequences SEQ ID Nos. 8 to 11,
in Brachypodium, chosen from the peptide sequences SEQ ID Nos. 50 and 51,
in rice, chosen from the peptide sequence SEQ ID No. 58, and
in poplar, chosen from the peptide sequences SEQ ID Nos. 63, 67 and 68.

13. A recombinant vector comprising an expression cassette as defined in claim 7.

14. A plant cell, comprising a recombinant vector as defined in claim 8.
